(19) United States
(12) Patent Application Publication
(10) Pub. No.: US 2018/0207020 A1
(43) Pub. Date: Jul. 26, 2018

(54) BREATHING ASSISTANCE APPARATUS

(71) Applicant: Oventus Medical Limited, Indooroopilly (AU)

(72) Inventors: Christopher Patrick HART, Eight Mile Plains (AU); Michael Leigh SLATER, Southport (AU); Zoran MILIJASEVIC, New South Wales (AU); Neil ANDERSON, Roseville (AU)

(21) Appl. No.: 15/750,023

(22) PCT Filed: Aug. 3, 2016
(86) PCT No.: PCT/AU2016/050696

§ 371 Date: Feb. 2, 2018

(30) Foreign Application Priority Data
Aug. 3, 2015 (AU) 2015903079
Nov. 30, 2015 (AU) 2015904963

(51) Int. Cl. A61F 5/56

(52) U.S. Cl. CPC A61F 5/566

(57) ABSTRACT

An apparatus for providing breathing assistance includes a body for positioning within an oral cavity of a user, the body defining at least one first opening for allowing airflow between lips of the user. Two second openings are provided in the oral cavity to allow air flow into and out of a posterior region thereof. Two channels each connect a respective second opening to the at least one first opening. Each channel passes at least one of at least partially along the buccal cavity and at least partially between the teeth to thereby provide an airway at least partially bypassing the nasal passage to act to replicate a healthy nasal passage and pharyngeal space. A tongue retaining portion includes a cavity for receiving a portion of a tongue, the tongue retaining portion configured to retain the tongue in an extended position to project at least partially between the teeth.

### BACKGROUND OF THE INVENTION

The present invention relates to apparatus for providing breathing assistance, and in particular an apparatus for providing breathing assistance during sleeping or exercise, as well as a method of manufacturing a breathing assistance apparatus.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Poor quality or ineffective breathing is an issue which can affect the performance of people in their day to day activities either while they are awake and/or when they are asleep. While awake this can be less optimal performance in activities such as sport or even while performing everyday tasks. While asleep breathing disorders can lead to snoring and/or sleep apnea.

Snoring arises due to vibration of soft tissues within the respiratory pathways of an individual, and is typically caused by obstructed air movement during breathing while sleeping. Snoring can arise from a range of different physical causes such as blocked sinuses, and typically occurs when the muscles of the upper throat relax during sleep.

Snoring can also be associated with Obstructive Sleep Apnoea (OSA), which is caused by obstruction of the upper airway and results in repetitive pauses in breathing during normal sleep. Individuals having OSA often suffer from daytime sleepiness and fatigue associated with significant levels of sleep disturbance, whilst a partners sleep patterns are also often disturbed by associated snoring.

Current therapy for treatment of OSA can include lifestyle changes, the use of mechanical devices, such as oral or nasal devices that augment the airway, surgical procedures to enlarge and stabilize the airway during sleep, and continuous or variable positive airway pressure (CPAP, VPAP) devices.

However, surgical procedures can be severe and are not therefore widely used unless absolutely necessary. Whilst CPAP and VPAP devices have had a positive impact, these can be uncomfortable to wear for prolonged time periods, are expensive, and are often noisy, which can in turn lead to additional sleep disturbance. As a result, surgery, VPAP and CPAP treatment have limited application in treating sleep apnoea, and are not generally considered appropriate treatment for snoring.

In terms of other mechanical devices, nasal devices have been used that dilate the nasal airway using traction or splinting. However, these have typically not had much success and can be uncomfortable for a user.

US2004/194787 describes an anti-snoring device that includes a flexible hollow tube for insertion into the user's mouth, having proximal and distal ends and an outer perimeter. The tube includes an extraoral segment at its proximal end, an intraoral segment at its distal end and an intermediate segment extending therebetween. The extraoral and intraoral segments each include at least one opening. The extraoral segment is for extending beyond the user's outer lips, the intermediate segment is of a sufficient length for extending along the buccopharyngeal pathway of the user's mouth, and the intraoral segment is of a sufficient length for extending beyond a retromolar space in the user's mouth, into the oropharynx and terminating between the posterior tongue and the soft palate. The anti-snoring device also includes a stop extending from the outer perimeter of the tube on the intraoral segment for securing the intraoral segment within the user's oropharynx. However, whilst this arrangement can assist in providing an additional airway, and hence reduce snoring and apnoea events, it can be uncomfortable to wear and can move within the mouth during use, which can reduce device effectiveness and in turn lead to additional breathing problems.

US2005/150504 describes a device which is removably insertable in the mouth for facilitating breathing while sleeping which provides a clear unobstructed airway by protrusive positioning of the mandible and/or delivery of pressurized air to the back of the mouth. The device has upper and lower tooth-contacting members and an airway defined between them, and is designed specifically for use with CPAP machines. Consequently, this device can only be used in limited circumstances, where CPAP machines are available, and is only used in the treatment of sleep apnoea.

WO2012/155214 describes an apparatus for providing breathing assistance, the apparatus including a body including a recess for receiving teeth of a user to thereby position the body within an oral cavity of the user, a first opening extending beyond lips of a user to allow air from outside the oral cavity to be drawn in through the opening, a second opening provided in the oral cavity to allow air to be directed into a posterior region of the oral cavity and a channel connecting the first and second openings, the channel extending through at least part of a buccal sulcus of the user.

It is also known to provide mouth guards for use during sport. For example, US2013/0074851 describes a dental appliance including arms disposed about occlusal pad to secure the dental appliance in a removable fashion to the teeth of the user. The occlusal pad is formed from an occlusal pad material transformable between a pliable state and a non-pliable state, in various aspects. Associated methods of use are also disclosed herein.

US2013/0081640 describes an interchangeable mouthguard component system. The system includes a mouthguard base with a receiving recess positioned within at least a front surface of the base, a securely attachable mouthguard component positioned to fit within the recess, one or more attaching posts positioned on one of the recess or component and one or more holes positioned opposite the posts on either the recess or component. The mouthguard component, when attached, is positioned sufficient to maintain a substantially flush front surface of the base.

However, mouth guards for use in sport are not adapted to provide assistance with breathing and can in some circumstances make breathing more difficult.

WO2012/140021 describes a method for generating a virtual orthodontic element for use in manufacturing an orthodontic appliance for a patient. The method comprises obtaining a patient data set for said patient, the patient data set comprises a virtual 3D teeth model, where said virtual 3D teeth model comprises a virtual upper jaw and a virtual lower jaw resembling the upper jaw and lower jaw, respectively, of the patient's mouth, arranging the virtual upper jaw and the virtual lower jaw in an initial relative configuration in a virtual articulator which is able to simulate the articulation between the virtual upper jaw and the virtual lower jaw based at least on motion relative to at least one axis representing the terminal hinge axis of the patient, designing the virtual orthodontic element based on at least a part of the virtual 3D teeth model and the arrangement of the 3D teeth model in the virtual articulator.

However, use of the virtual articulation means that the resulting orthodontic element is not necessarily optimised for patient comfort as this may not take into account the particular articulation of the patient's jaws.

### SUMMARY OF THE PRESENT INVENTION

In one broad form the invention seeks to provide an apparatus for providing breathing assistance, the apparatus including:
a) a body for positioning within an oral cavity of a user, the body defining:
   i) at least one first opening for allowing airflow between lips of the user;
   ii) two second openings provided in the oral cavity to allow air flow into and out of a posterior region of the oral cavity; and,
   iii) two channels, each channel connecting a respective second opening to the at least one first opening and each channel passing at least one of at least partially along the buccal cavity and at least partially between the teeth to thereby provide an airway for the user, the airway at least partially bypassing the nasal passage to thereby act to replicate a healthy nasal passage and pharyngeal space; and,
b) a tongue retaining portion including a cavity for receiving a portion of a tongue of the user, in use, wherein the tongue retaining portion is configured to retain the tongue in an extended position to project at least partially between the teeth of the user.

Typically retention of the tongue in the cavity is assisted by an at least partial vacuum created within the cavity.

Typically the tongue retaining portion is integral with the body.

Typically the body includes two first openings, each first opening connected to a respective second opening, and wherein the tongue retaining portion is disposed between the two first openings.

Typically the tongue retaining portion is secured to the body.

Typically at least part of the first opening extends into an anterior portion of the oral cavity and the tongue retaining portion extends at least partially through the first opening.

Typically the tongue retaining portion at least partially occludes the first opening and portions of the first opening disposed about opposing sides of the tongue retaining portion, permitting airflow into the channels connected to the respective second openings.

Typically the tongue retaining portion is sealingly engaged with the body so as to preclude airflow into and out of the anterior portion of the oral cavity.

Typically the tongue retaining portion is an elliptical bulb structure.

Typically the first opening is substantially elliptical.

Typically the height of the first opening is in the range of 5 to 10 mm.

Typically the width of the first opening is in the range of 20 to 25 mm.

Typically the first opening is substantially elliptical.

Typically each channel at least one of:
a) includes a first channel portion extending through the user's buccal cavity and a second channel portion in fluid communication with the first channel portion and extending between the user's maxillary and mandibular teeth;
b) not contained within the buccal cavity;
c) extends substantially entirely between the user's maxillary and mandibular teeth;
d) includes a first channel portion extending inside of the user's teeth and a second channel portion in fluid communication with the first channel portion and extending between the user's maxillary and mandibular teeth; and,
e) includes a channel portion extending between the user's maxillary and mandibular teeth and no channel portion extending through the user's buccal cavity.

Typically a first channel portion extending through the user's buccal cavity has a substantially semi-elliptical cross section and the second channel portion extending between the user's maxillary and mandibular teeth has a substantially rectangular cross section, the second channel portion extending laterally inward from the first channel portion.

Typically at least one of the cross sectional shape and cross sectional area of at least one of the first and second channel portions varies from the first opening to the second opening.

Typically the second channel portion has a substantially rectangular cross section extending laterally between the user's maxillary and mandibular teeth in use, and wherein the first channel portion extending inside of the user's teeth has a substantially arcuate shape, extending from an inward end of the second channel portion at least one of:
a) upwardly and inwardly; and,
b) downwardly and inwardly.

Typically a shape and size of the channels varies in accordance with an anatomy of the oral cavity of the user.

Typically the second openings are positioned over the last or back tooth on each side of the top jaw.

Typically the body is made of at least one of:
a) metal;
b) titanium alloys;
c) high strength polymers; and,
d) cobalt chromium alloys.

Typically the body is made using additive manufacturing.

Typically the body is coated with at least one of:
a) a medical grade polymer;
b) a medical grade elastomer;
c) silicone;
d) polyurethane;
e) epoxy; and,
f) parylene.

Typically the apparatus includes at least one insert, the insert being positioned at least partially between the user's teeth and the body in use.

Typically the insert is customised for a user's teeth.

Typically the insert is at least one of removable and replaceable.

Typically the insert is attached to the body using at least one of:
a) an adhesive; and,
b) mechanical engagement between the insert and body.

Typically the body includes at least one side wall extending from a lateral face and a side wall lip projecting inwardly from at least part of the at least one side wall, the at least one insert engaging the lip to thereby couple the insert to the body.

Typically the body includes a face lip projecting from at least part of the lateral face, the insert engaging the face lip and side wall lip to thereby couple the insert to the body.

Typically the body includes upper and lower faces and corresponding upper and lower side walls, each channel including a first channel portion positioned between an outer side wall and the upper and lower side walls, and a second channel portion extending laterally inwardly from the first channel portion between the upper and lower faces.

Typically the apparatus includes a plurality of inserts for each user, each insert being adapted to provide a different positioning of at least one of the body and the user's teeth.

Typically the insert is made of at least one of:
a) metals;
b) ceramics;
c) a polymer;
d) polyether block amide;
e) polyvinylsiloxane;
f) polyurethane; and,
g) ethylvinylacetate.

Typically the insert is made by at least one of:
a) by additive manufacturing; and,
b) by laser sintering.

In one broad form the invention seeks to provide a method for manufacturing a breathing assistance apparatus for a user, the method including:
a) providing a body for positioning within an oral cavity of the user, the body including:
   i) at least one first opening for allowing airflow between lips of the user;
   ii) two second openings provided in the oral cavity to allow air flow into and out of a posterior region of the oral cavity;
   iii) two channels, each channel connecting a respective second opening to a respective first opening and each channel passing at least one of at least partially along the buccal cavity and at least partially between the teeth to thereby provide an airway for the user, the airway at least partially bypassing the nasal passage and acting to replicate a healthy nasal passage and pharyngeal space; and;
b) providing a tongue retaining portion including a cavity for receiving a portion of a tongue of the user, in use, wherein the tongue retaining portion is configured to retain the tongue in an extended position to project at least partially between the teeth of the user.

Typically the body is created using additive manufacturing and is made of at least one of:
a) metal;
b) titanium alloys;
c) high strength polymers; and,
d) cobalt chromium alloys.

Typically the method includes applying a coating to the body.

Typically the method coating is applied to inner surfaces of the body.

Typically the method includes applying the coating to the body by at least one of:
a) dip coating;
b) spray coating; and,
c) vapour coating.

Typically the method includes applying primers to the body prior to coating.

Typically the method includes polishing at least part of the body using at least one of mechanical and electrochemical polishing.

Alternatively the body is moulded and is made of at least one of:
a) acrylic; and,
b) polymer.

Typically the method includes:
a) obtaining shape information indicative of a shape of the user's oral cavity; and,
b) manufacturing the breathing assist apparatus using the shape information.

Typically the method includes deriving the shape information from at least one of:
a) an impression;
b) a series of photos;
c) a scan;
d) a CT scan;
e) a 3D scan of the user's teeth; and,
f) cone beam imaging.

Typically the series of photos of the patient's mouth or impression are taken with a smart phone and the photos are then loaded into a software program to derive a 3D image including an STL file.

Typically the shape information includes dimensions of the oral cavity of the user.

Typically the method includes:
a) selecting one of a number of standard bodies in accordance with the shape information; and,
b) using the shape information, at least one of:
   i) modifying the selected standard body; and,
   ii) creating at least one insert.

Typically the method includes:
a) obtaining template data representing a body design;
b) modifying the body design using the information derived from the scan;
c) generating modified template data using the modified body design; and,
d) manufacturing the body using the modified template data.

Typically the modified template data is in the form of a print file for use in an additive manufacturing machine.

Typically the tongue retaining portion is secured to the body using an adhesive.

Typically the tongue retaining portion is secured to the body using at least one of an adhesive and mechanical engagement between the insert and body.

In one broad form the invention seeks to provide a method for manufacturing a breathing assistance apparatus for a user, the method including using additive manufacturing to create a body for positioning within an oral cavity of the user, the body including:
a) two first openings for allowing airflow between lips of the user;
b) two second openings provided in the oral cavity to allow air flow into and out of a posterior region of the oral cavity;
c) two channels, each channel connecting a respective second opening to a respective first opening and each channel passing at least one of at least partially along the buccal cavity and at least partially between the teeth to thereby provide an airway for the user, the airway at least partially bypassing the nasal passage and acting to replicate a healthy nasal passage and pharyngeal space; and;
d) a tongue retaining portion including a cavity for receiving a portion of a tongue of the user, wherein the tongue retaining portion is disposed between the two first openings.

In one broad form the invention seeks to provide a method for use in manufacturing a breathing assistance apparatus for a user, the method including:
a) determining a desired jaw position;
b) obtaining shape information indicative of a shape of at least the user's teeth with the user's jaws in the desired jaw position;
c) manufacturing a body of a breathing assistance apparatus at least in part using the shape information; and,
d) manufacturing at least one insert for the desired jaw position of the user, the insert being positioned at least partially between the user's teeth and the body in use.

In one broad form the invention seeks to provide a method for use in manufacturing an oral appliance, the method including:
a) determining a desired jaw position;
b) obtaining shape information indicative of a shape of the user's teeth with the user's jaws at least in the desired jaw position; and,
c) manufacturing the oral appliance at least in part using the shape information.

In one broad form the present invention seeks to provide apparatus for providing breathing assistance, the apparatus including:
a) a body for positioning within an oral cavity of a user, the body defining:
   i) at least one first opening for allowing airflow between lips of the user;
   ii) two second openings provided in the oral cavity to allow air flow into and out of a posterior region of the oral cavity;
   iii) two channels, each channel connecting a respective second opening to a respective first opening and each channel passing at least partially between the teeth and not within the buccal cavity, to thereby provide an airway for the user, the airway at least partially bypassing the nasal passage and acting to replicate a healthy nasal passage and pharyngeal space.

Typically each channel includes a first channel portion extending inside of the user's teeth and a second channel portion in fluid communication with the first channel portion and extending between the user's maxillary and mandibular teeth.

Typically the second channel portion has a substantially rectangular cross section extending laterally between the user's maxillary and mandibular teeth in use, and wherein the first channel portion extending inside of the user's teeth has a substantially arcuate shape, extending from an inward end of the second channel portion at least one of:
a) upwardly and inwardly; and,
b) downwardly and inwardly.

It will be appreciated that the broad forms of the invention and their respective features can be used independently and/or in conjunction and reference to these as separate forms of the invention is not intended to be limiting.

### BRIEF DESCRIPTION OF THE DRAWINGS

An example of the present invention will now be described with reference to the accompanying drawings, in which: —

FIG. 1A is a schematic underside perspective view of a first example of an apparatus for providing breathing assistance;

FIG. 1B is a second schematic underside perspective view of the apparatus of FIG. 1A;

FIG. 1C is a schematic plan view of the apparatus of FIG. 1A;

FIG. 1D is a schematic cross sectional view along the lines A-A′ of FIG. 1C;

FIG. 1E is a schematic cross sectional view along the lines B-B′ of FIG. 1C;

FIG. 1F is a schematic cross sectional view along the lines C-C′ of FIG. 1C;

FIG. 1G is a schematic cross sectional view along the lines D-D′ of FIG. 1C;

FIG. 1H is a schematic cross sectional view along the lines E-E′ of FIG. 1C;

FIG. 1I is a schematic cross sectional view along the lines F-F′ of FIG. 1C;

FIG. 2A is a schematic topside perspective view of a third example of an apparatus for providing breathing assistance;

FIG. 2B is a schematic plan view of the apparatus of FIG. 2A;

FIG. 2C is a schematic cross sectional view along the line A-A′ of FIG. 2B;

FIG. 3 is a flow chart of an example of a method for manufacturing a breathing assistance apparatus;

FIG. 4A is a schematic topside perspective view of a further example of an apparatus for providing breathing assistance;

FIG. 4B is a schematic topside perspective view of the apparatus of FIG. 4A including a tongue retaining portion;

FIG. 4C is a schematic plan view of the apparatus shown in FIG. 4B;

FIG. 4D is a schematic front view of the apparatus shown in FIG. 4B showing the tongue retaining portion in hatched lines.

FIG. 4E is a schematic cross sectional view along the lines A-A′ of FIG. 4C showing the cavity of the tongue retaining portion;

FIG. 4F is a schematic cross sectional view along the lines B-B′ of FIG. 4C showing a representation of a portion of a user's tongue retained, in use;

FIG. 5A is a schematic perspective topside front view of a further example of an apparatus for providing breathing assistance;

FIG. 5B is a schematic perspective topside rear view of the apparatus of FIG. 5A;

FIG. 5C is a schematic side view of the apparatus of FIG. 5A;

FIG. 5D is a schematic plan view of the apparatus of FIG. 5A;

FIG. 5E is a schematic front view of the apparatus of FIG. 5A;

FIG. 5F is a schematic rear view of the apparatus of FIG. 5A;

FIG. 5G is a schematic perspective topside front view of the apparatus of FIG. 5A with the tongue retaining insert removed;

FIG. 5H is a schematic perspective topside front view of the tongue retaining insert of FIG. 5A;

FIG. 5I is a schematic side view of the tongue retaining insert of FIG. 5H;

FIG. 5J is a schematic plan view of the tongue retaining insert of FIG. 5H;

FIG. 6A is a schematic frontside perspective view of a further example of an apparatus for providing breathing assistance;

FIG. 6B is a schematic rearside perspective view of the apparatus of FIG. 6A;

FIG. 6C is a schematic rear view of the apparatus of FIG. 6A;

FIG. 6D is a schematic plan view of the apparatus of FIG. 6A;

FIG. 6E is a schematic cross sectional view along the lines A-A′ of FIG. 6D;

FIG. 6F is a schematic cross sectional view along the lines B-B′ of FIG. 6D;

FIG. 6G is a schematic cross sectional view along the lines C-C′ of FIG. 6D;

FIG. 7A is a schematic rearside perspective view of a further example of an apparatus for providing breathing assistance;

FIG. 7B is a schematic plan view of the apparatus of FIG. 7A;

FIG. 7C is a schematic cross sectional view along the lines A-A′ of FIG. 7A;

FIG. 7D is a schematic cross sectional view along the lines B-B′ of FIG. 7A;

FIG. 7E is a schematic cross sectional view along the lines C-C′ of FIG. 7A;

FIG. 8A is a schematic rearside perspective view of a further example of an apparatus for providing breathing assistance;

FIG. 8B is a schematic plan view of the apparatus of FIG. 8A;

FIG. 8C is a schematic cross sectional view along the lines A-A′ of FIG. 8A;

FIG. 8D is a schematic cross sectional view along the lines B-B′ of FIG. 8A; and,

FIG. 8E is a schematic cross sectional view along the lines C-C′ of FIG. 8A.

### DETAILED DESCRIPTION OF THE PREFERRED EMBODIMENTS

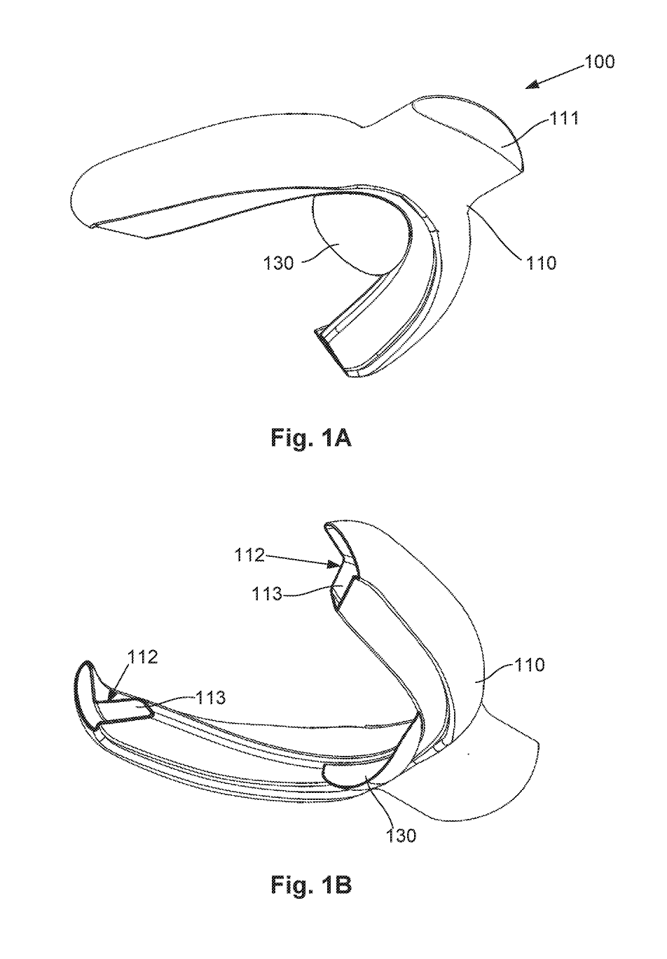

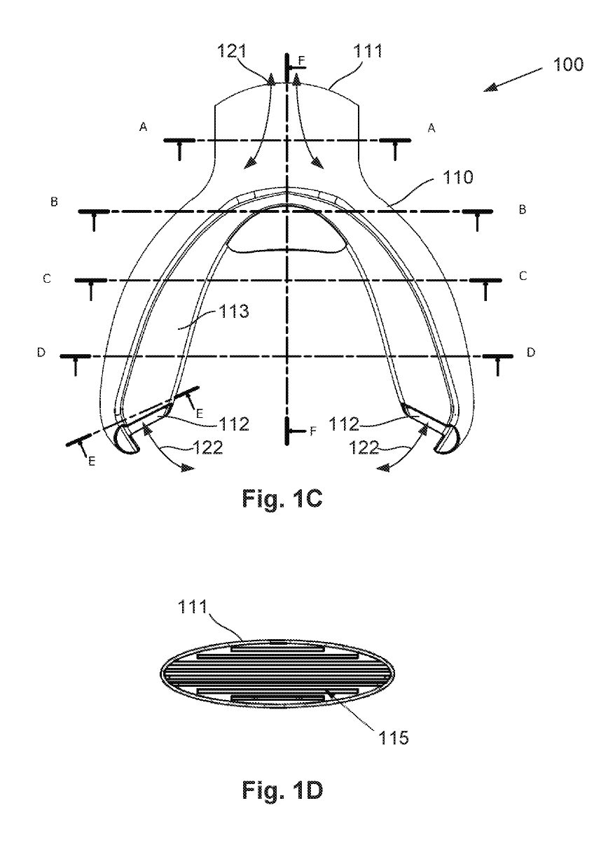

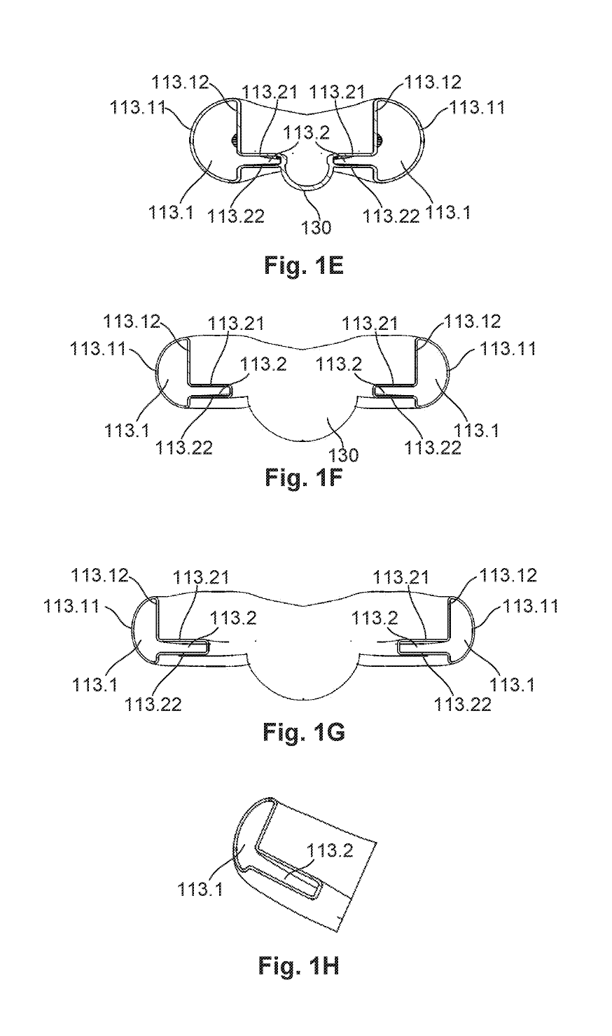

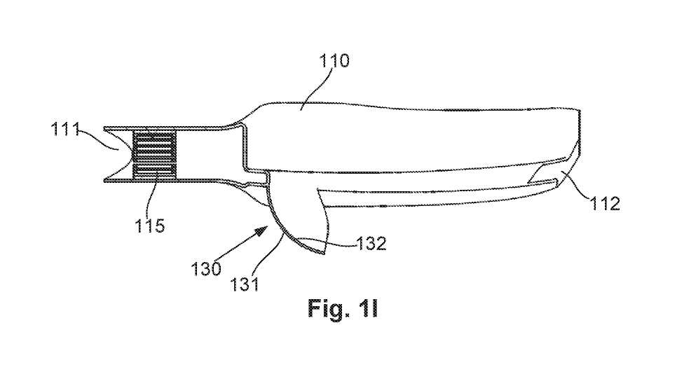

An example of a breathing assist apparatus will now be described with reference to FIGS. 1A to 1I.

In this example, the apparatus 100 includes a body 110 for positioning within an oral cavity of a user. The body 110 includes at least one first opening 111 for allowing airflow between lips of the user and two second openings 112 provided in the oral cavity to allow air flow into and out of a posterior region of the oral cavity. Two channels 113 are provided, each channel connecting a respective second opening 112 to the first opening 111 and each channel 113 passing at least one of at least partially along the buccal cavity and at least partially between the teeth to thereby provide an airway for the user, the airway at least partially bypassing the nasal passage and acting to replicate a healthy nasal passage and pharyngeal space.

As a result of this arrangement, during inhalation, air can enter the apparatus 100 via the first opening 111, as shown by the arrows 121, pass along the channels 113, to the second openings 112. The second openings 112 are typically provided towards a posterior of the user's oral cavity, on a lingual side of the user's teeth, so that air is directed into the posterior of the oral cavity, as shown by the arrows 122. In one particular example, the channels 113 and second openings 112 are provided so that air flows through the user's hammular notch. During exhalation, airflow is reversed, as will be appreciated by persons skilled in the art.

Thus, the apparatus provides an oral appliance for providing breathing assistance. This can be used during sleep, for example for the treatment of both snoring and sleep apnea, and can also be used at other times, for example during exercising to assist with maintaining optimum airflow, in the treatment of respiratory conditions, such as emphysema, and to assist in supplying air, for example during surgery, CPR (Cardiopulmonary resuscitation), or the like. The device has a first opening 111, which can extend beyond the lips, or at least keep the lips apart, to allow airflow therethrough. Air passes through airways defined by the channels 113, on each side of the oral cavity, and is directed into a posterior region of the mouth through second openings 112, bypassing issues with tongue and lower jaw position. In more severe cases, the first opening 111 can be connected to an external device, such as a CPAP (Continuous Positive Airway Pressure) machine, air supply, or the like providing more comfort and increased patient compliance compared to a mask.

Providing air flow directly into a posterior portion of the user's oral cavity has a number of benefits. In particular, this avoids obstructions created by the nasal cavity, soft palate and tongue which can lead to snoring and apnoea events, and helps reduce the drying effects of air flow, which can in turn lead to user discomfort. This makes the apparatus comfortable to wear whilst ensuring an unobstructed air flow thereby preventing snoring and apnoea events. Thus, for example, nasal obstructions can be bypassed by air flow through the apparatus, thereby bypassing the nasal airway or adding to it in the case of a partial obstruction. Furthermore, air flowing below or on both sides of the soft palette helps prevent collapse of the soft palate, which can in turn lead to additional obstruction.

Additionally, during sports, the apparatus can act to provide protection to the user's teeth, for example by cushioning and/or absorbing impacts, whilst also providing for improved airflow.

In one example, the body is manufactured using additive manufacturing, such as a 3D printing process. This is particularly beneficial as it allows the channel arrangement 113 to be made, whilst minimising the thickness of the channel walls. This helps maximise the cross sectional area of the channels, thereby assisting airflow, whilst minimising overall device volume, thereby helping to maintain comfort. For example, the use of additive manufacturing allows a body having channel wall thicknesses of less than 0.5 mm and more typically approximately 0.3 mm or less, although it will be appreciated that other thicknesses can be accommodated if required. Thus, this significantly reduces the volume/bulk compared to an acrylic device made using standard processes, thereby maximising the available airway size, whilst also leading to more user comfort and improved compliance. However, this is not essential and alternatively, the device can be a moulded device, formed from acrylic or another suitable polymer, for example using injection moulding techniques or the like.

A number of further features will now be described.

In one example, for a device adapted to be used at rest, for example during sleeping or sitting, each channel has a cross sectional area of at least one of at least 10 mm², at least 20 mm², at least 30 mm², at least 40 mm² and at least 50 mm². Additionally, the at least one of the first opening and/or the second openings have a cross sectional area of at least one of at least 50 mm², at least 70 mm², at least 90 mm², at least 100 mm² and at least 110 mm². The dimensions selected will vary depending on a wide range of factors, including whether the device is required to provide a partial or complete airway, for example to bypass a partial or complete blockage. Additionally, this will depend on the intended use and the associated airflow requirements. Typically the dimensions of the channels 113 and/or openings 111, 112 are selected so that in conjunction with the user's existing airways, the total airway available corresponds to the cross sectional area of an airway in a healthy subject for both nasal and pharyngeal airways.

In any event, from the above it will be appreciated that the cross sectional areas used will depend on the preferred implementation and intended use, so for example, a smaller cross sectional area may be used for children, adolescents, or individuals with only partial obstructions. In contrast increased cross sectional areas may be used where a high flow rate is required, for example in the event that the device is to be used to provide breathing assistance during exercise.

The volume of air inhaled will vary depending on the level of activity. The following table is an indication of a male at rest, exercising and an elite sportsman. Clearly the volume of air required increases which is a result of both more breaths per minute and the volume of air in each breath.

| | | | Elite athlete |
|---|---|---|---|
| | Male - at rest | Male exercising | exercising |
| Amount of air per | 8 | 60 | 200 |
| minute - litres | | | |
| Breaths per minute | 12 | 40 | 60 |
| Volume per breath - | 0.533 | 1.5 | 3.33 |
| litres | | | |
| Ratio compared to | 1 | 3 | 6 |
| male at rest | | | |

Consequently the size of the airway for the present invention will increase for a device that is to be used for aerobic activities. For general exercise this will be 2-3 times the area for use at rest and for elite sports 5-6 times the area.

As a result, for a device adapted to be used during exercise, each channel has a cross sectional area of at least one of at least 20 mm²; at least 40 mm²; at least 60 mm²; at least 80 mm²; at least 100 mm²; at least 150 mm²; at least 200 mm²; at least 250 mm²; and, at least 300 mm². Similarly, at least one of the first opening and the second openings have a cross sectional area of at least one of at least 100 mm²; at least 140 mm²; at least 180 mm²; at least 200 mm²; at least 220 mm²; at least 330 mm²; at least 440 mm²; and at least 550 mm². It will of course be understood that different channel and opening dimensions could therefore be determined on a case by case basis depending on the intended usage.

The channels can have a wide variety of configurations and may be sized and shaped depending on the anatomy of the oral cavity of the user. This is typically done to maximise the available airway, whilst ensuring comfort for the user. In one example, this is achieved by measuring the oral cavity of the user, for example by taking dental impressions, a series of photos, or scans of the user's teeth and/or oral cavity and then customising the apparatus based on the measured size, as will be described in more detail below. In general however, the apparatus will have a number of common features irrespective of the user.

In one example, the channel includes two interconnected channel portions, including a first channel portion 113.1 extending through the buccal cavity, between the user's cheeks and teeth, and a second channel portion 113.2 in fluid communication with the first channel portion and extending between the user's maxillary and mandibular teeth. It will be appreciated that the channel portions are integrally formed and reference to them as separate portions is largely for the purpose of illustration. In any event, this arrangement maximises the cross sectional area of the channels 113, whilst maintaining comfort for the user, by distributing the airway between the user's teeth and cheeks and between the user's teeth. In particular, this avoids the second channel portion 113.2 between the teeth being too high, which would result in the mouth being held open too far, whilst also avoiding the first channel portions 113.1 causing the cheeks to bulge.

In one example, the first channel portion 113.1 has substantially semi-elliptical cross section and the second channel portion 113.2 has a substantially rectangular cross section, the second channel portion 113.2 extending laterally inwardly from the first channel portion 113.1. The use of a semi-elliptical cross section for the first channel portion, allows the curved outer surface 113.11 to lie against the inner surface of the user's cheek, avoiding sharp edges being in contact with the cheeks, and thereby ensuring comfort. The linear inner surface 113.12 can rest against the teeth, thereby maximising the volume of the first channel portion. The second channel portion 113.2 has a rectangular cross section, allowing the teeth to rest against the upper and lower faces 113.21, 113.22 of the second channel portion 113.2 in use. However, other channel configurations could be used, for example in the event that the channel does not extend into the buccal region and examples of alternative configurations will be described in more detail below.

In one example, the cross sectional area of the first and second channel portions vary between the first and second openings 111, 112. For example, in the arrangement of FIGS. 1A to 1I, the first channel portion progressively decreases from the first opening 111 to the second opening 112, whilst the cross sectional area of the second channel portion 113.2 progressively increases from the first opening 111 to the second opening 112. This allows the overall cross section of the channel 113 to be maintained, whilst having the channel portions 113.1, 113.2 conform as far as possible to the natural space available in the oral cavity. It will be appreciated that any variation can be used, depending for example on the configuration of the user's oral cavity.

Thus, the airways defined by the channels 113 have a cross section this is shaped to conform to a persons oral cavity, and in particular the available space between the maxillary and mandibular teeth, as well as between the teeth and the cheeks. In one example, the first opening 111 has an elliptical shape at the air entrance between the lips and also a semi-elliptical shape (vertical) along the gum line to the first molar on each side and then smoothly transition to an “L” shaped cross section from the first molar to the rear of the appliance, so that at least some of the airway is between the user's teeth in this area.

In one example, the first opening is removably mounted to the body. In this regard, this allows different styles of first opening to be used, for example to interface with an external device or the like, as well as allowing different sizes of first opening to be used to suit user requirements. This also allows for easy cleaning of the opening and/or replacement of filters, heat/moisture exchangers, valves or the like provided therein, as will be described in more detail below. The first opening can be made of any suitable material and could include plastic, metals, ceramics or the like and could also be made using different materials to the body.

In one example, the second openings are angled inwardly at between 10° and 50°, more typically between 20° and 40° and preferably about 30° to assist in airflow into and out of the oral cavity, and in particular to direct airflow towards the centre of the oral cavity. Additionally and/or alternatively, the second openings are positioned over the last or back tooth on each side of the top jaw.

As mentioned above, the body 110 is typically made using additive manufacturing, which in one example is used to create a body made of metal and in particular a titanium alloy and/or cobalt chromium alloy, although it will be appreciated that any suitable material may be used, including high strength polymers, plastics, VeroGlaze (MED620) dental material, or the like. The body can be coated with a medical grade polymer and in one example, a medical grade elastomer, such as silicone or polyurethane, epoxy or parylene, for improved comfort as well as ensuring biocompatability. In one example, the coating can include an Active Composite Guidance, which is a 3 dimensional composite resin with different shapes and sizes and which can be bonded to the body to ensure accurate positioning of the body with respect to the user's teeth. Coatings can be applied to the body using any suitable technique, such as dip coating, vapour coating, or spray coating the body, thereby ensuring all exposed surfaces, including internal surfaces of the channels, are coated. As part of this process, this can include applying primers to the body prior to coating, thereby ensuring the coating adheres to the body.

As an alternative, or in addition to coating, at least part of the body can be polished using at least one of mechanical and electrochemical polishing.

In one example, the apparatus includes a filter 115 for filtering air flowing through the apparatus. This can help remove particulates, pollen or other contaminants entrained in air flowing into the device, which can assist in reducing respiratory irritation, which can in turn exacerbate snoring and breathing difficulties. The filter 115 can be positioned anywhere within the body 110, but is typically provided within the first opening 111, thereby allowing this to be easily removed and replaced if required. The filter could be of any suitable form and could include a porous plastic or cloth based filter, and may include additional materials for added functionality. For example, the filter can also include activated carbon for filtering out pollution/bacteria.

Additionally and/or alternatively, a heat/moisture exchanger can be provided that controls the water and temperature content of the air being inhaled by exchanging heat and moisture with exhaled air. Examples of such exchangers can be found for example in U.S. Pat. No. 5,433,192, and these will not therefore be described in any further detail.

Additionally or alternatively, the apparatus can include a valve (not shown) for regulating air flow through the apparatus. In one example, this can be used to resist outflow of air from the second openings to the first opening. This can assist in regulating breathing and in particular allow for rapid inhalation, whilst ensuring slower exhalation, thereby optimising gas exchange within the lungs, for example to minimise the chances of hyperventilation. The valve can be of any suitable form, such as a ball valve, umbrella valve, or the like, and can be adjustable or titratable to ensure that the level of flow control is appropriate to the user.

The body can include a lingual flange 130 for engaging mandibular teeth to thereby maintain mandibular position. In this regard, the lingual flange 130 includes a first face 131, which in use, engages the user's mandibular incisors, thereby controlling the relative positions of the maxillary and mandibular jaws. In this regard, it is known that temporomandibular joint disorder (TMD) arises when the upper and lower jaws are misaligned. This may be naturally occurring or can result from injury, or the like. Regardless, such jaw misalignment tends to contribute to airway obstructions by changing the shape of the upper airway, and moving the tongue towards the posterior of the oral cavity, which can in turn exacerbate issues associated with OSA and snoring. Accordingly, providing the lingual flange 300 at an appropriate location allows the jaws of the user to be aligned thereby reducing the effects of TMD, and hence further reducing the likelihood of snoring and OSA. As will be described in more detail below, the apparatus can further include an insert that can be adapted to cover the first face of the lingual flange, with different thicknesses of the insert being provided to allow for thereby adjustment of mandible position in defined increments.

In addition, a second side 132 of the lingual flange can be provided to define a pocket for receiving the user's tongue. In this regard, the second side 132 is of a concave shape, so that when the tongue abuts against the lingual flange 132 a suction effect is created, thereby helping to retain the user's tongue towards the anterior of the oral cavity, which in turn helps further reduce airway obstruction caused by the position of the user's tongue.

Thus, in the current example, the lingual flange 130 is rounded and faces downwards and rearwards at an appropriate angle, such as 45° to suit the shape of the lingual region under the tongue for the bottom side as well as the top to comfortably allow the tongue to be positioned.

In one example, the lingual flange is movably mounted to the body to thereby allow adjustment of a user's mandibular position. Whilst this can be achieved in any suitable manner, in one example, the lingual flange 130 includes a flange recess for receiving a flange mounting projecting from a lower surface of the body 110. A screw is provided extending through the lingual flange and the flange mounting, so that as the screw is rotated the relative position of the flange mounting within the flange recess is adjusted, thereby progressively moving the flange. It will therefore be appreciated that movably mounting the flange 130 to the body 110 allows the relative degree of mandibular advancement to be adjusted to thereby provide an optimum outcome for the user.

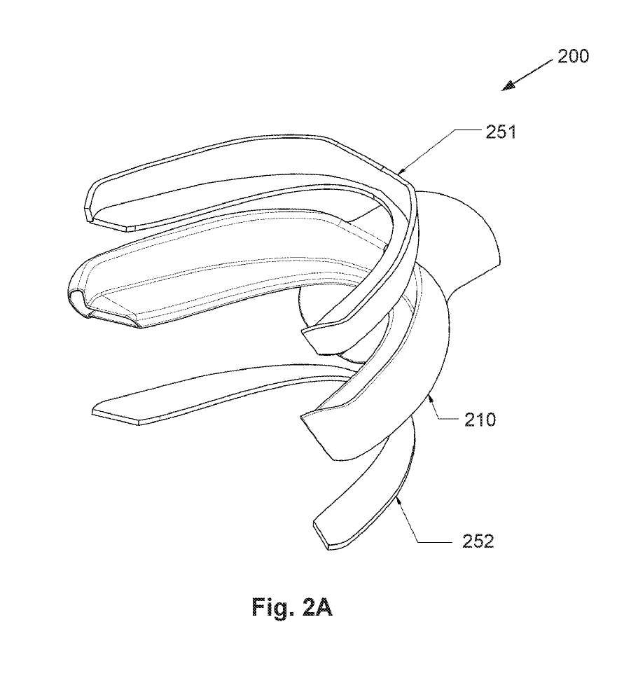

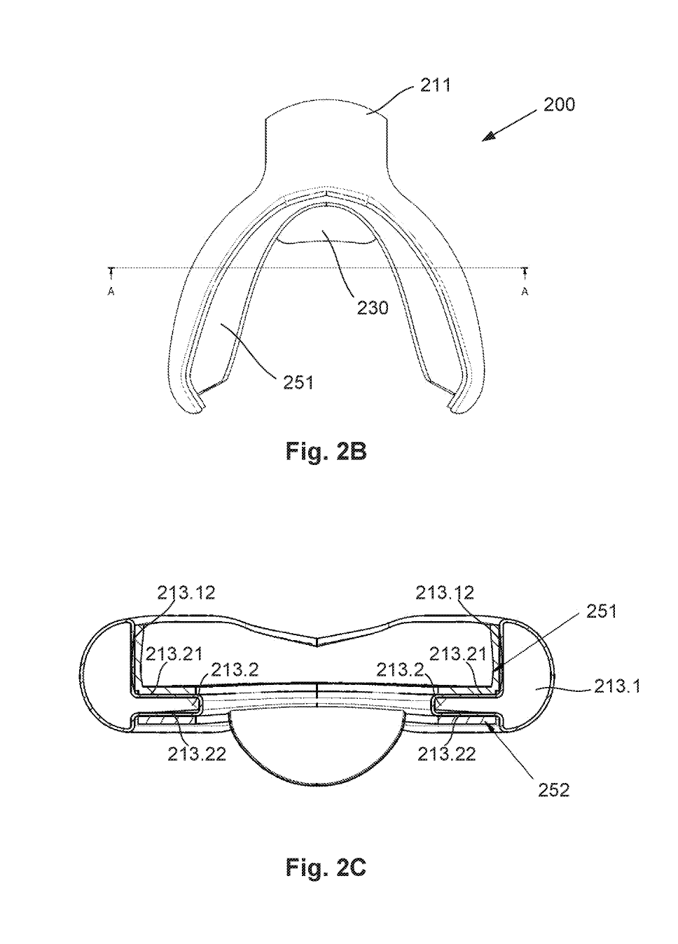

A second example apparatus is shown in FIGS. 2A to 2C. In this example, similar reference numbers increased by 100 are used to refer to similar features.

In this example, the apparatus 200 includes at least one, and more typically two inserts 251, 252, the inserts being positioned between the user's teeth and the body 210 in use. The inserts generally have an arcuate shape and “L”-shaped cross section so that they conform to the shape of the channels. In particular, an upper insert 251 is adapted to abut against the upper face 213.21 of the second channel portion 213.2, whilst a lower insert 252 abuts against the linear inner surface 213.12 of the first channel portion 213.1 and the lower face 213.22 of the second channel portion 213.2.

The insert(s) are typically formed from acrylic, polyvinylsiloxane, polyurethane or ethylvinylacetate, polyether block amide, another suitable polymer or the like, and are optionally customised for a user's teeth, thereby maximising comfort of the device in use. The inserts can be removable or replaceable, allowing a plurality of different inserts to be provided with each insert being adapted to provide a different positioning of at least one of the body and the user's teeth. The inserts could also be coupled to the body, for example using an adhesive or other suitable technique. The inserts could be manufactured using any appropriate technique, such as moulding, and/or additive manufacturing and in particular selective laser sintering of polyether block amide or the like.

In one particular example, the apparatus includes an insert having a lingual flange layer extending over at least part of the lingual flange and wherein a thickness of the lingual flange layer is used to adjust the user's mandibular position. Thus, the apparatus can include multiple inserts for each user, and wherein each insert has a different lingual flange layer thickness for adjusting the user's mandibular position in known increments.

The inserts can also be adapted to provide impact protection for example for use in sports applications.

When manufacturing the above described apparatus, this typically involves creating a custom apparatus for each user. In order to achieve this, the method typically includes obtaining shape information indicative of a shape of the user's oral cavity and then manufacturing the breathing assist apparatus using the shape information.

The shape information can include dimensions of the oral cavity of the user and can be derived from a scan, such as a CT scan of the user's oral cavity, or alternatively can be obtained from dental impressions, 3D models, 3D scans of the user's teeth, cone beam imaging, moulds, digital impressions of an intra-oral scan, or the like. Alternatively, the dimensions could be obtained from a series of photos of the patient's mouth or impressions taken with a smart phone or the like.

As part of this process, the method can involve selecting one of a number of standard bodies in accordance with the shape information and using the shape information, at least one of modifying the selected standard body and creating at least one insert. Thus, a range of standard template bodies can be provided, with these being modified as required in order to prepare a custom body for each user. Thus, for example, a range of template devices, such as 6 to 10 designs can suit 80-90% of patients which can then be selected and then individually customised by the dentist or dental laboratory for each patient.

The modification/customisation can be performed by adding custom inserts and additionally or alternatively custom manufacturing or finishing of the body. Custom manufacturing can include obtaining template data representing a body design, modifying the body design using the information derived from the shape information, generating modified template data using the modified body design and then manufacturing the body using the modified template data. In one particular example, the modified template data is in the form of a print file for use in an additive manufacturing machine. Thus, for example, this process can be used to generate a custom STL (STereoLithography) print file, thereby allowing a body 110 to be custom printed for each individual. Thus, for example, a series of photos could be loaded into a software program to derive a 3D image, which is then used to generate an STL file. However, alternatively, customisation can be a post manufacture process of shaping using convention techniques.

As part of the above manufacturing process, additional components, such as the mandibular advancement mechanisms or inserts can be included in the STL file and hence “printed” into the device. Alternatively, the inserts could be manufactured separately and hence be included in a separate STL file, and then attached to the body in a separate process, for example using an adhesive of mechanical coupling process, such as a friction fit, interference fit, or the like.

In one particular example, the manufacturing process uses additive manufacturing of titanium using an STL file which is a merging of the device design features adapted to suit scans of the patient's mouth or scans of an impression of their teeth, although alternatively a limited number of standard shapes could be used, with these being customised by hand or machining using standard techniques, or through the use of separate additional custom inserts.

In one example, when the body 110 is produced it is cleaned and then coated with a suitable polymer material such as a medical grade elastomer eg: silicone or polyurethane or polymers such as epoxy. This can be achieved using a dipping or spray process either by hand into a container of the solution or using dedicated equipment such as those available from DipTech Systems Inc in the USA. The use of dip or spray coating solutions allow for the coating of the inner areas of the airways. Primers may be used to optimise adhesion or the natural surface roughness of the device does provide mechanical attachment. Examples of the coatings include but are not limited to: MED16-6606 RTV Silicone Dispersion from Nusil Technology of 1050 Cindy Lane, Carpinteria, Calif. 93013; Baymedix SD for seamless polyurethane films from Bayer MaterialScience AG of 51368 Leverkusen Germany. 301-2 and 302-3M for epoxy or cyanoacrylate coatings from Epoxy Technology, Inc. of 14 Fortune Dr. Billerica, Mass. 01821

In another example, when the body 110 is produced it is cleaned and then coated with a suitable polymer material such as a medical grade polymer from the vapour form such as parylene. Such equipment and material can be supplied by Specialty Coating Systems Ontario, Calif. USA.

In another example, when the body 110 is produced it is polished using mechanical and/or electrochemical means. This may be with abrasive bits and polishing pads using hand or bench tools. It may also be achieved using specialised abrasive and electrochemical treatment from companies such as from Best in Class (BinC) industries in St Priest France

As previously described, a mandibular advancement arrangement can be incorporated. In one example, this is in the form of a fixed lingual flange 130 printed as part of the body 110. Alternatively, if an adjustable mechanism is required, the lingual flange 130 is provided as a separate component which is positioned on a track with a screw adjustment. Alternatively, arms (metal or plastic) can be attached to the upper airway appliance and then to the occlusal region of the mandibular teeth. These can be easily fitted by the patient using screws or other mechanical attachment methods

In any event, the above described apparatus provides a dental insert that provides an airway running from between the lips into the buccal sulcus and/or between the teeth then into the area over or behind the wisdom teeth, through the hamular notch and then opening into a region near or just off the soft palate. This provides an alternative airway, helping mitigate the impact of partial or total obstructions either in the nasal passages, soft palate or created by the tongue, thereby reducing the impact of such obstructions, and hence preventing snoring and OSA.

The provision of a lingual flange can be used to move a user's mandible forward, helping to prevent obstruction by the tongue, thereby maintaining a clear glossopharyngeal airway, with the degree of advancement being controlled depending on severity of symptoms. For example, this can be to the extremity of the neuromuscular zone of tolerance or beyond if necessary to clear tongue obstruction. The concave inner surface of the tongue flange, together with an optional tongue recess, can also act as a suction cup to hold the tongue forward.

In addition to this, the apparatus can assist in maintaining alignment of the upper and lower jaws which can reduce the impact of TMD.

The apparatus may also include a screw inserted into the lingual flange allowing the apparatus to be titratable for use with PSG (Polysomnograph) and EMG (Electromyograph).

It will be appreciated from the above described examples, that the use of an airway running from the lips into the buccal sulcus and/or between the teeth then into the area over or behind the rear teeth or wisdom teeth, through the hamular notch and then opening into a region near or just off the soft palate could be implemented as part of existing insert that includes a body insertable into the oral cavity. Accordingly, the above described examples are for the purpose of illustration only and are not intended to be limiting.

Accordingly, it will be appreciated that the above device can treat individuals with sleep apnea (especially more severe cases) but typically is more comfortable than and hence will have greater compliance rates compared to a mask. In this regard, it has been shown that 30-50% of CPAP users are non-compliant/non-users within 2 years of starting their treatment and further recent studies have shown that mouth breathing is an indication for non compliance.

The apparatus can assist in significantly improving sleep as shown by both sleep studies and general home use (quality of sleep and daytime function) with or without a CPAP. The apparatus overcomes the issues of dry mouth and ideally reduces the number of cases of excessive salivation with initial use. The device is more comfortable to wear leading to lower side effects such as teeth rubbing, teeth movement, jaw misalignment, or perceived “mouth noise” with movement. Additionally, the apparatus does not cause any damage to dentine layer of the teeth, has lower costs to manufacture and therefore when and if required, can be profitable if current selling prices are reduced, and complies with regulatory requirements.

However, whilst the above description has focussed on applications of sleep apnoea and snoring, use of the apparatus is not so limited and can apply in any scenario where breathing assistance is required.

For example, the apparatus can be used in sports applications to deliver more air into athletes lungs and allow them to breathe more easily during training/exercise. In these cases the airways may be larger but the fundamental design is the same. The apparatus can be used in general well being where people have a blocked nose from a cold or allergies, as well as to assist in delivery of other therapy such as steam to help alleviate congestion and airway irritation, for example, from asthma. In this regard, it will be appreciated that the use of an in-built filter can help reduce irritation from pollutants, whilst the use of a control valve can assist in regulating breathing, thereby improving breathing efficiency, and mitigating the effects of breathing difficulties. The valve can also control the rate of air when breathing in and then slow down for breathing out to help balance the oxygen and CO₂ exchange in the lungs and reduce the chances of hyperventilation or lactic acid build up in athletes. The device can also be used for treating pulmonary or respiratory disorders such as COPD (Chronic Obstructive Pulmonary Disease) or emphysema, to replace the use of a mask or be in addition to other airway devices for example during administration of oxygen, air, anaesthetic, CPR or the like, as well as for other day to day activities.

It will also be appreciated from the above that the apparatus could form part of an orthodontic or dental appliance, in which case the airway could be integrated into a body also having other functionality.

It will also be appreciated that the inserts could be made to have a gap or space between the teeth and the walls of the insert which would allow teeth whitening gel to be used while wearing the device.

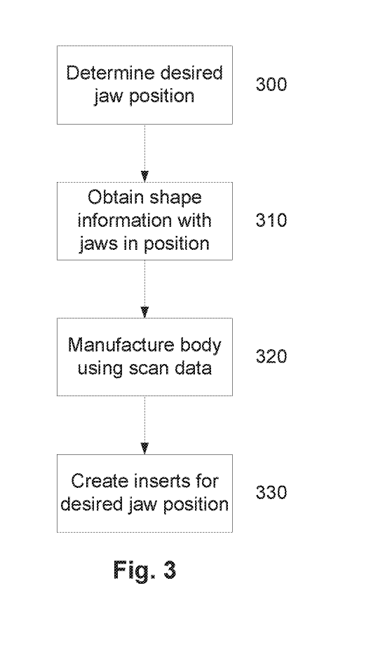

An example of a method for use in manufacturing a breathing assistance apparatus for a user, and in particular for manufacturing a custom breathing assistance apparatus will now be described with reference to FIG. 3.

In this example, at step 300, the method includes determining a desired jaw position. In this regard, a desired jaw position is taken to be the jaw position that will be assumed by the user when the breathing assistance apparatus is being worn and in particular a jaw position that is comfortable and optimises airflow for the user, thereby minimising snoring or other undesirable breathing events.

The desired jaw position will typically vary for each user depending on a range of different factors, such as occlusal vertical dimension, tongue size, inter-arch relationship, tooth alignment, jaw joint function, soft tissue condition, lymphatic tissue, periodontal status, Maxillary Bone architecture, arch form, palatal vault. Accordingly, it is typical to determine a desired jaw position that is likely to clear glossopharyngeal obstruction while maintaining comfort.

Thus, the desired jaw position typically allows for the maxillary and mandibular teeth of the user to be spaced apart to a sufficient degree to accommodate the breathing assistance apparatus and optionally with the lower jaw advanced to move the tongue forward within the oral cavity.

Following this, at step 310 shape information indicative of a shape of the user's teeth with the user's jaws in the desired jaw position is obtained. This can be achieved in any appropriate manner as previously described, and this could include obtaining a bite impression, which is subsequently scanned, but more typically includes scanning the user's teeth and jaws, while the jaws are in the desired jaw position, for example using Cone Beam Computed Tomography (CBCT), CT scans, digital impressions of an intra-oral scan, or the like. Alternatively, this could be achieved using a 3-D imaging technique or the like as will be described in more detail below. Thus the desired jaw position determined at step 300 is typically recorded, for example using a bite registration material, positioning spacer, or the like, with this being used during a subsequent scanning process allowing the shape information to be obtained whilst the jaws are in the desired jaw position.

At step 320, a body of a breathing assistance apparatus is manufactured at least in part using the shape information. This could include creating a custom designed body, and typically includes modifying an STL print file or the like, of a standard template body, in accordance with the shape information.

At step 330, at least one insert is manufactured for the desired jaw position of the user, allowing the insert to be positioned at least partially between the user's teeth and the body in use, to thereby make the apparatus more comfortable to wear. This could be achieved in any suitable manner, and typically involves curing a resin once it has been moulded into a desired shape.

Accordingly, the above described process involves placing the user's jaws into desired jaw positions before shape information is determined. The shape information can then be used to manufacture the body of a breathing assistance apparatus, or other oral implant, ensuring that this is appropriately configured for the user, and in particular to ensure the user's jaws are held in the desired jaw position, so that breathing is optimised. Further details are described in copending application WO2015/149127, the contents of which are incorporated herein by reference.

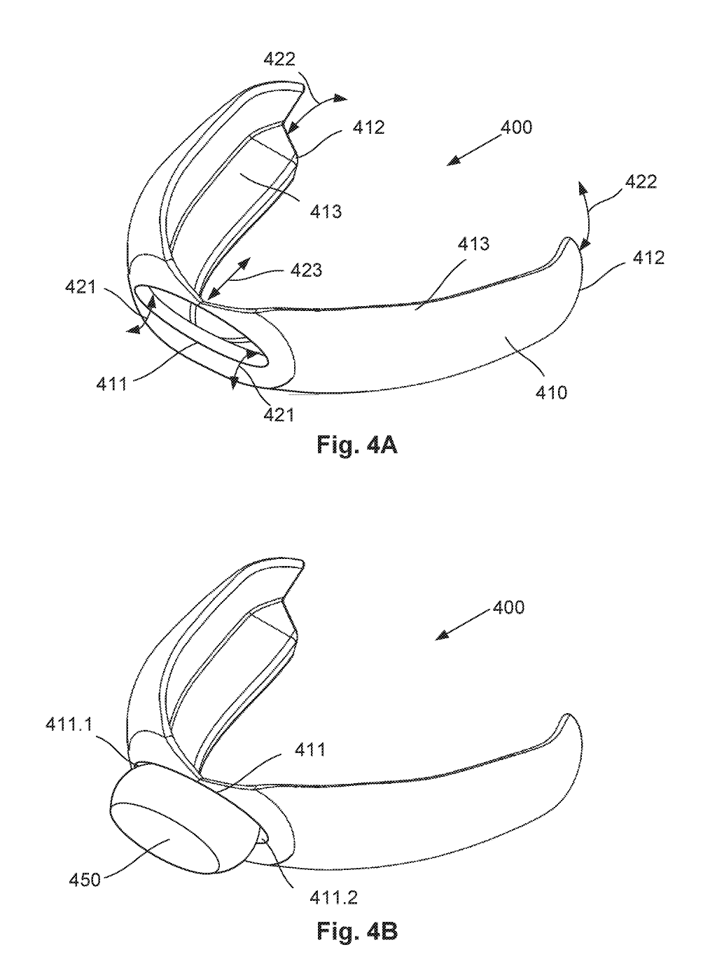

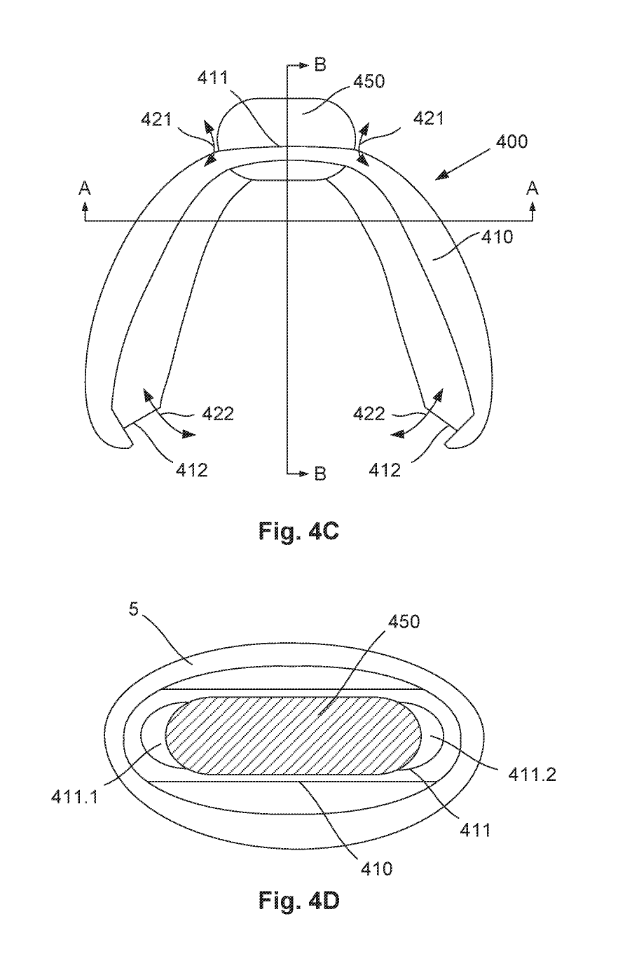

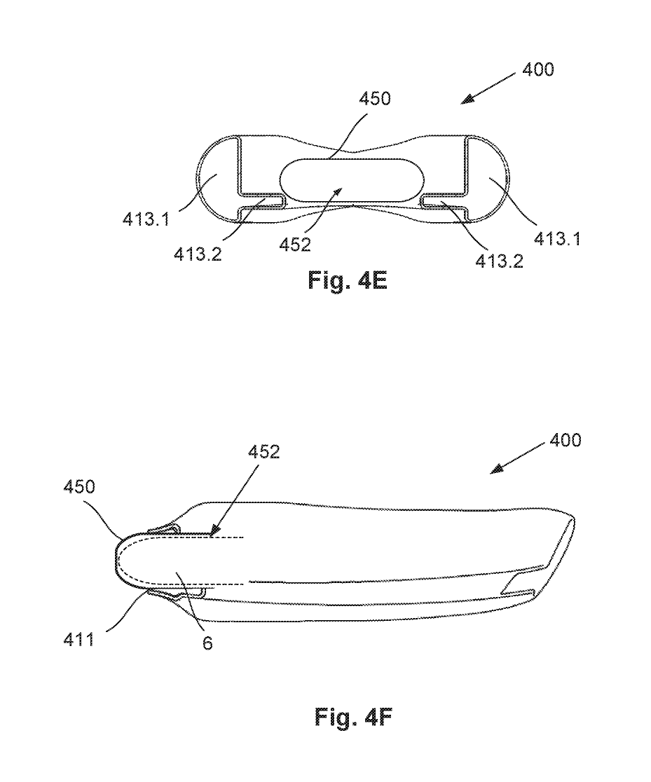

Referring now to FIGS. 4A to 4F, a further example of an apparatus for providing breathing assistance shall be described.

In the example shown in FIG. 4A, the apparatus 400 includes a body 410 for positioning within an oral cavity of a user. The body 410 defines at least one first opening 411 for allowing airflow 421, 423 between lips of the user. The at least one first opening 411 extends into an anterior region of the oral cavity (not shown) so as to provide an airway for the user.

Similar to previously described examples, the body 410 includes two second openings 412 provided in the oral cavity to allow air flow 422 into and out of a posterior region of the oral cavity. Additionally, the body 410 includes two channels 413, each channel 413 connecting a respective second opening 412 to a respective first opening 411 and each channel 413 passing at least one of at least partially along the buccal cavity and at least partially between the teeth to thereby provide an auxiliary airway for the user, the auxiliary airway at least partially bypassing the nasal passage and acting to replicate a healthy nasal passage and pharyngeal space.

Typically each channel 413 includes a first channel portion 413.1 extending through the buccal cavity, between the user's cheeks and teeth, and a second channel portion 413.2 in fluid communication with the first channel portion and extending between the user's maxillary and mandibular teeth.

As shown in FIG. 4A, the first opening 411 is a through-opening, extending through a front portion of the body and into the anterior region of the oral cavity. This allows air to flow directly into a front portion of the mouth whilst also flowing into and out of a posterior region of the oral cavity through openings 412. The additional airway provided by airflow 422 through the first opening 411 may be particularly beneficial during exercise (e.g. aerobic sports and activity) when additional air intake is necessary.

In one example, the apparatus 400 may include one-way valves (not shown) in the auxiliary airways to modify the rate at which air is expelled by the user. The one-way valves can be used to resist outflow of air from the second openings 412 to the first opening 411. This can assist in regulating breathing and in particular allow for rapid inhalation, whilst ensuring slower exhalation, thereby optimising gas exchange within the lungs, for example to minimise the chances of hyperventilation. The one-way valves can be of any suitable form, such as a ball valve, umbrella valve, or the like, and can be adjustable or titratable to ensure that the level of flow control is appropriate to the user. In this example, air is able to be breathed in through the airway created by the first opening 411 as well as through the auxiliary airways. However, upon exhalation, due to the one-way valves, air is only able to be expelled through first opening 411 and not through the auxiliary airways thereby allowing for more or faster inhalation and less or slower exhalation.

In one example, the first opening 411 is substantially elliptical having a height in the range 5 to 4 mm, and more particularly in the range selected from 5 to 6 mm, 6 to 7 mm, to 8 mm, 8 to 9 mm and 9 to 4 mm. The width of the first opening 411 is typically in the range 20 to 25 mm, and more particularly in the range selected from 20 to 21 mm, 21 to 22 mm, 22 to 23 mm, 23 to 24 mm and 24 to 25 mm.

The apparatus 400 shown in FIG. 4A may be adapted as shown in FIGS. 4B to 4F to include a tongue retaining portion 450 including a cavity 452 for receiving a portion of a tongue 6 of a user, in use.

In this example, the tongue retaining portion 450 is configured to retain the tongue in an extended position to project at least partially between the teeth and optionally further between lips 5 of the user. By bringing the tongue further forward and retaining it in that position, the likelihood of the tongue obstructing the airway is significantly reduced. This may be particularly beneficial for people who have a large tongue or a tongue prone to falling back in the oral cavity and obstructing the airway with or without mandibular advancement. Once the tongue is retained further forward in the extended position, the airway allows air to be delivered to the pharyngeal at the back of the mouth without significant tongue obstruction.

In one example, the tongue retaining portion 450 is a separate component that is secured to the body 410. Typically, the tongue retaining portion 450, and hence in use the tongue, extends at least partially through the first opening 411 and generally at least a portion of the tongue retaining portion 450 will protrude out beyond the first opening 411 in order to accommodate the extended position of the tongue.

As shown in FIG. 4B, typically the tongue retaining portion 450 at least partially occludes the first opening 411 and portions of the first opening 411.1, 411.2 disposed about opposing sides of the tongue retaining portion 450 permit airflow 421 into the channels 413 connected to the respective second openings 412. In other words, with the tongue retaining portion 450 in place within the first opening 411, there are spaces or air gaps either side of the tongue retaining portion which permit air to access the respective channels 413. In alternative arrangements, tubes may be connected to portions 411.1, 411.2 of the first opening 411 which are connected to a CPAP hose and CPAP machine.

In one example, the tongue retaining portion 450 is inserted through the first opening 411 and sealingly engaged with the body 410. In this way, airflow through the first opening 411 into and out of the anterior portion of the oral cavity is precluded and oral breathing is maintained through the auxiliary airways. The tongue retaining portion 450 may be sealed to the body 410 to form an airtight seal around at least an interior portion of the first opening 411 and secured thereto using any suitable adhesive.

The tongue retaining portion 450 is typically manufactured from any suitable medical grade polymer, such as be silicone, TPU (Thermoplastic Polyurethane), EVA (Ethylene-vinyl acetate) or other biocompatible elastomer/flexible plastic. In one example, the tongue retaining portion 450 is an elliptical bulb structure which is shaped to receive a tongue of the user and more generally is of a suitable size to conform to the profile of the first opening 411. The bulb structure is typically flexible so that a user can squeeze it with their fingers in order to form a vacuum seal around the tongue.

In operation, a user puts the apparatus into their mouth and positions their tongue in the cavity of the tongue retaining portion. The user may then squeeze and release the outside of the tongue retaining portion to create a partial vacuum in the cavity sufficient to prevent the tongue from being easily withdrawn. Typically therefore, retention of the tongue in the cavity is assisted by a vacuum created within the cavity by squeezing and releasing the bulb structure.

Whilst typically the tongue retaining portion is separate to the body of the apparatus and secured or fixed thereto, this is not essential. In other forms, the tongue retaining portion may be integral with the body and formed for example from an additive manufacturing or injection moulding process. Also, whilst typically the tongue retaining portion will protrude at least partially outward from the first opening, in other arrangements, the body may form a shroud around the tongue retaining portion so that the tongue retaining portion is contained wholly within the body structure.

In an example where the tongue retaining portion is integral with the body of the apparatus the body includes two first openings for allowing airflow between lips of the user. Each first opening is connected to a respective second opening, and the tongue retaining portion is disposed between the two first openings.

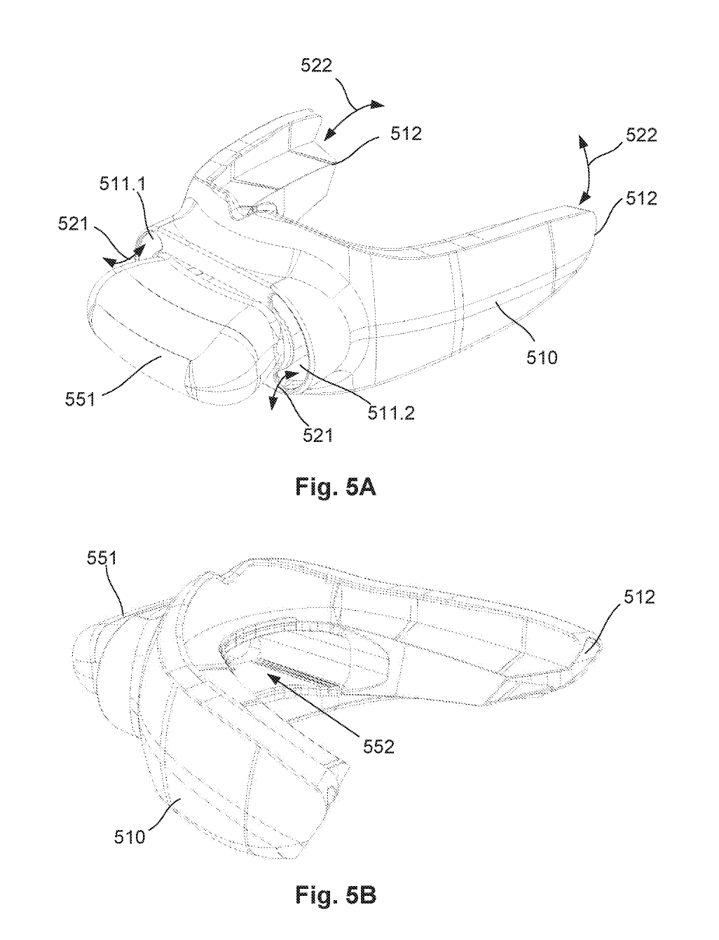

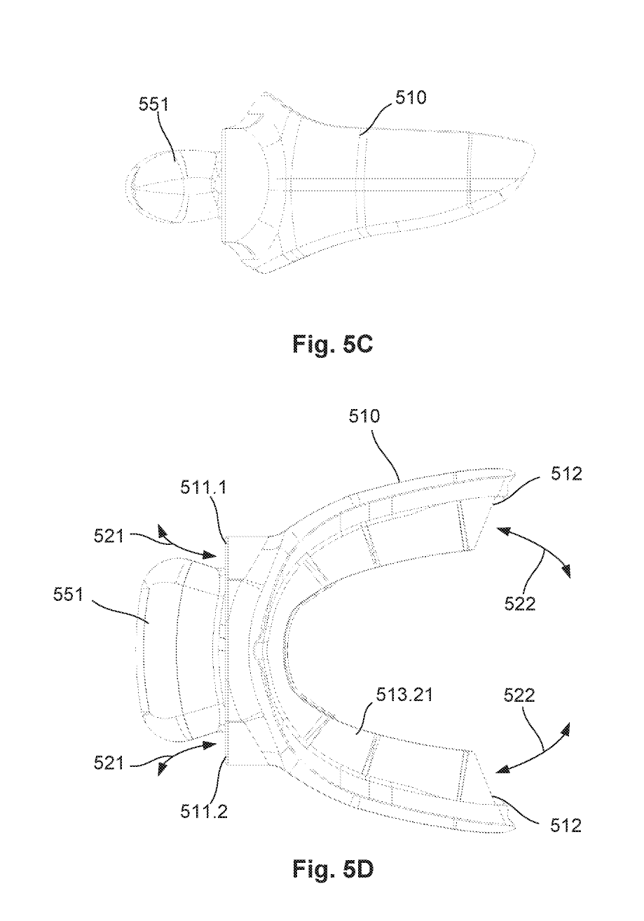

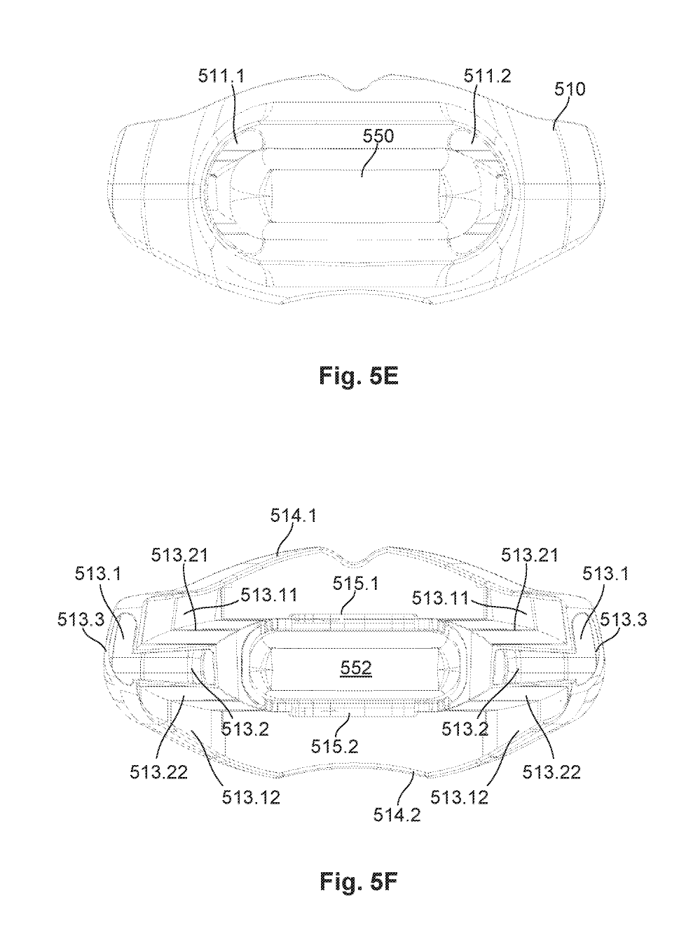

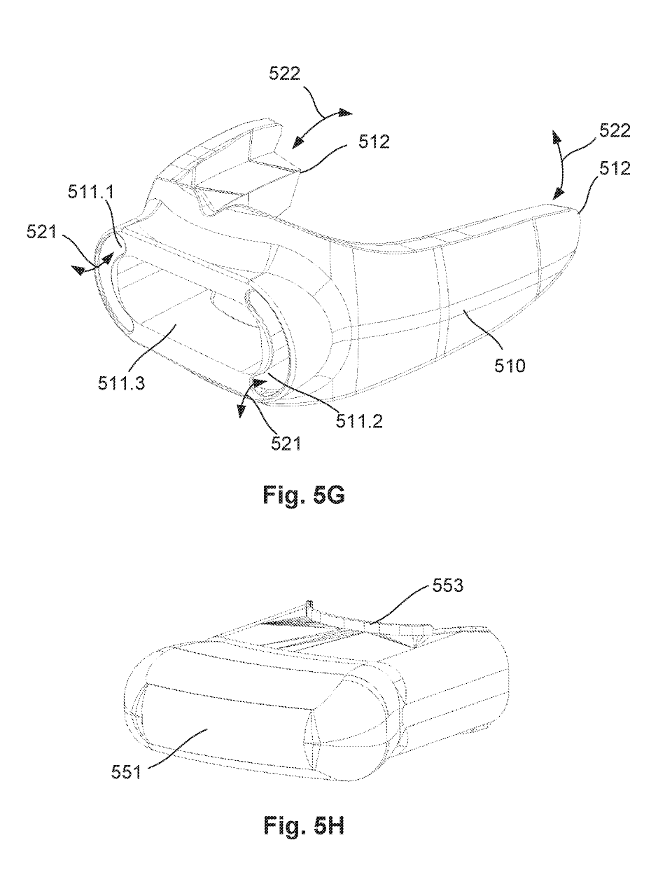

A further example tongue retaining arrangement will now be described in more detail with reference to FIGS. 5A to 5J.

In this example, the apparatus again includes a body 510 having two first openings 511.1, 511.2, and two second openings 512 provided in the oral cavity to allow air flow 522 into and out of a posterior region of the oral cavity. Two channels are provided extending between respective first and second openings, each channel including a first channel portion 513.1 extending through the buccal cavity, between the user's cheeks and teeth, and a second channel portion 513.2 in fluid communication with the first channel portion and extending between the user's maxillary and mandibular teeth.

In one example, the first channel portion 513.1 has substantially semi-elliptical cross section and is defined by upper and lower side walls 513.11, 513.12, each extending from a respective upper and lower lateral face 513.21, 513.22, and a curved outer side wall 513.3, whilst the lateral faces 513.21, 513.22 define a generally rectangular second channel portion 513.2.

In this example, an opening 511.3 is provided adapted to accommodate a tongue retaining insert 550 including a body 551 defining an internal cavity adapted to accommodate the user's tongue in use. The tongue retaining insert can include a flange 553 that abuts against the breathing apparatus body 510 when the tongue retaining insert 550 is provided in the opening 511.3, thereby sealing the opening 511.3 so that air flow occurs via the first and second openings 511.1, 511.2, 512 and channels.

The tongue retaining portion 550 is typically manufactured from any suitable medical grade polymer such as be silicone, TPU (Thermoplastic Polyurethane), EVA (Ethylene-vinyl acetate) or other biocompatible elastomer/flexible plastic. In one example, the tongue retaining portion 550 is an elliptical bulb structure which is shaped to receive a tongue of the user and which can be flexible so that a user can squeeze it with their fingers in order to form a vacuum seal around the tongue.

In operation, a user puts the apparatus into their mouth and positions their tongue in the cavity of the tongue retaining portion. The user may then squeeze and release the outside of the tongue retaining portion to create a partial vacuum in the cavity sufficient to prevent the tongue from being easily withdrawn. Typically therefore, retention of the tongue in the cavity is assisted by a vacuum created within the cavity by squeezing and releasing the bulb structure.

In this example, the breathing apparatus is again adapted to be used in conjunction with inserts similar to those shown in FIGS. 2A to 2C. In this example, the body 510 includes side wall lip 514.1, 514.2 projecting inwardly from a respective one of the upper and lower side walls 513.11, 513.12. In use, the insert (not shown) can engage the lip to thereby couple the insert to the body 510. Similarly a face lip 515.1, 515.2 can project from at least part of the upper or lower lateral face 513.21, 513.22, with the insert engaging both the face lip 515.1, 515.2 and side wall lip 514.1, 514.2 to thereby couple the insert to the body. Whilst the provision of lips has been described for both the upper and lower side walls and faces it will be appreciated that this is not essential and these could be provided only on upper or lower side walls, for example depending on the need to attach inserts to the body.

The tongue retaining arrangement described above can be used with the previous breathing apparatus examples described with respect to FIGS. 1 and 2, and it will therefore be appreciated that features of those examples could be used in conjunction with the tongue retaining arrangement.

Whilst the above arrangements have focussed on arrangements including at least part of a channel portion extending along a buccal cavity of the user, it will be appreciated that this is not essential, and the channel may alternatively extend wholly outside or not be contained within the buccal cavity. In this example, the channel can include a first channel portion extending inside of the user's teeth and a second channel portion in fluid communication with the first channel portion and extending between the user's maxillary and mandibular teeth. Alternatively, a single channel portion could extend only between the user's maxillary and mandibular teeth.

In one particular example, the channel includes a second channel portion that has a substantially rectangular cross section extending laterally between the user's maxillary and mandibular teeth in use, and a first channel portion extending inside of the user's teeth has a substantially arcuate shape, extending from an inward end of the second channel portion. The first channel portion could extend upwardly and inwardly, downwardly and inwardly or a combination of the two.

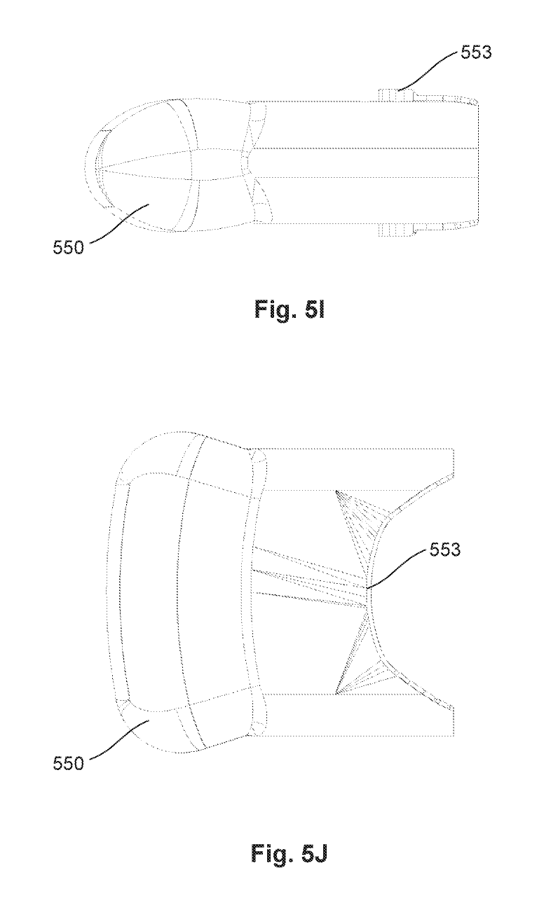

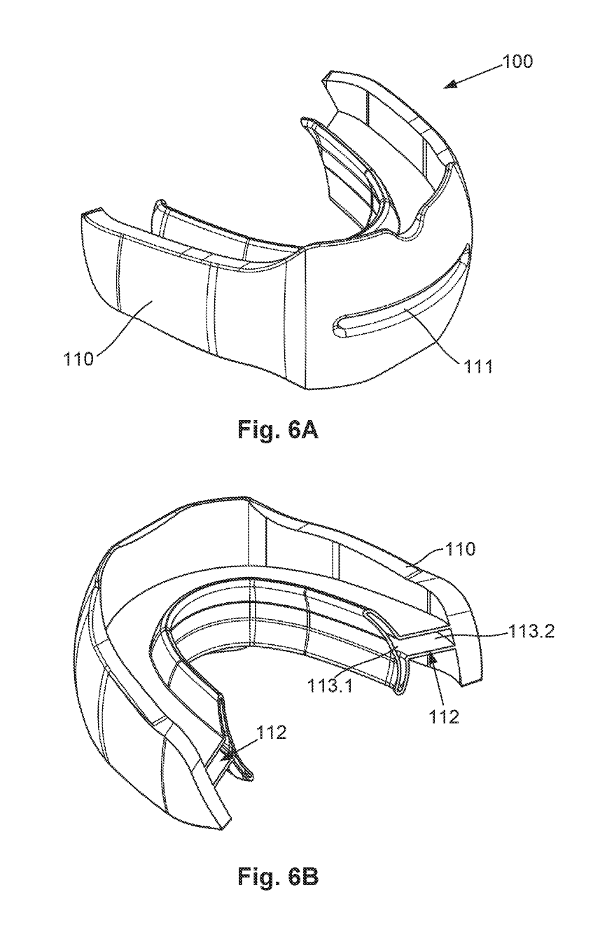

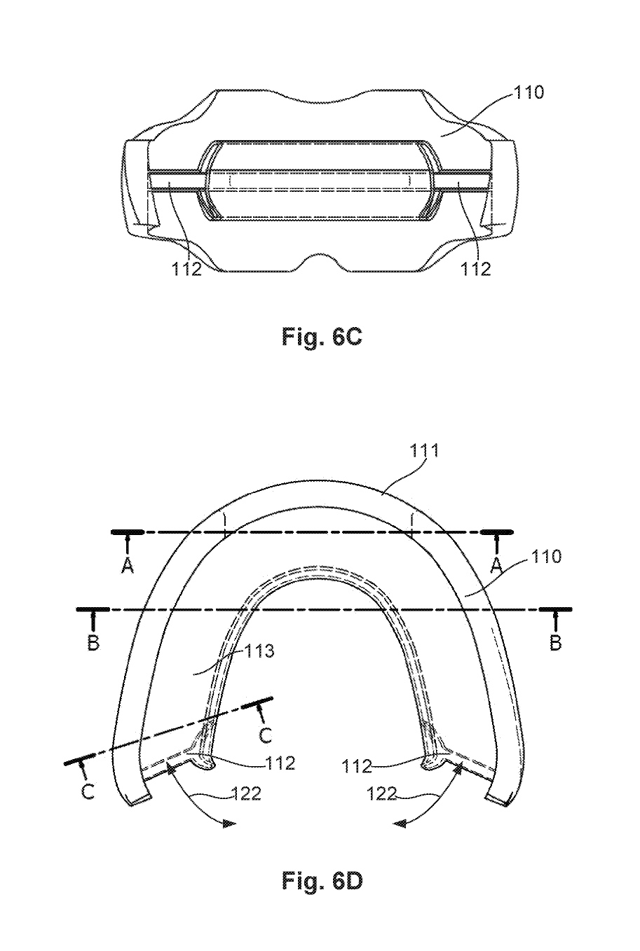

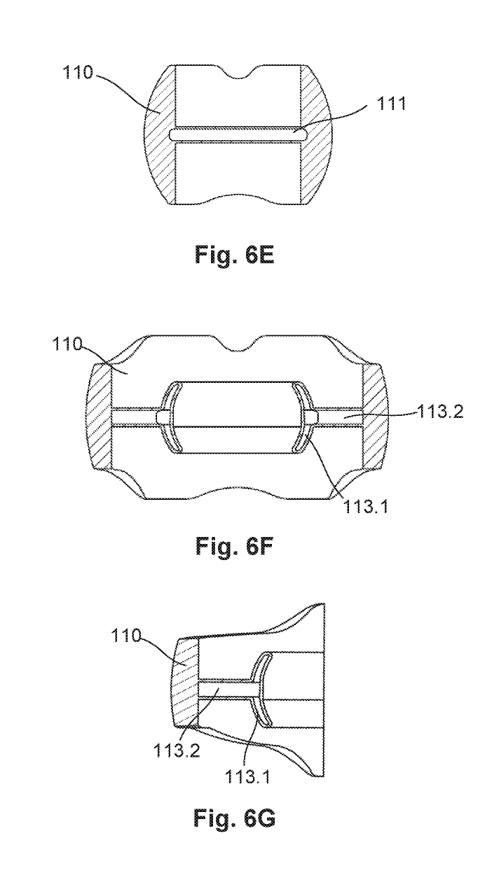

Example configurations are shown in FIGS. 6A to 6G, 7A to 7E and 8A to 8E. These examples use similar reference numerals to the arrangements of FIGS. 1A to 1H, and these features will not be described in detail.

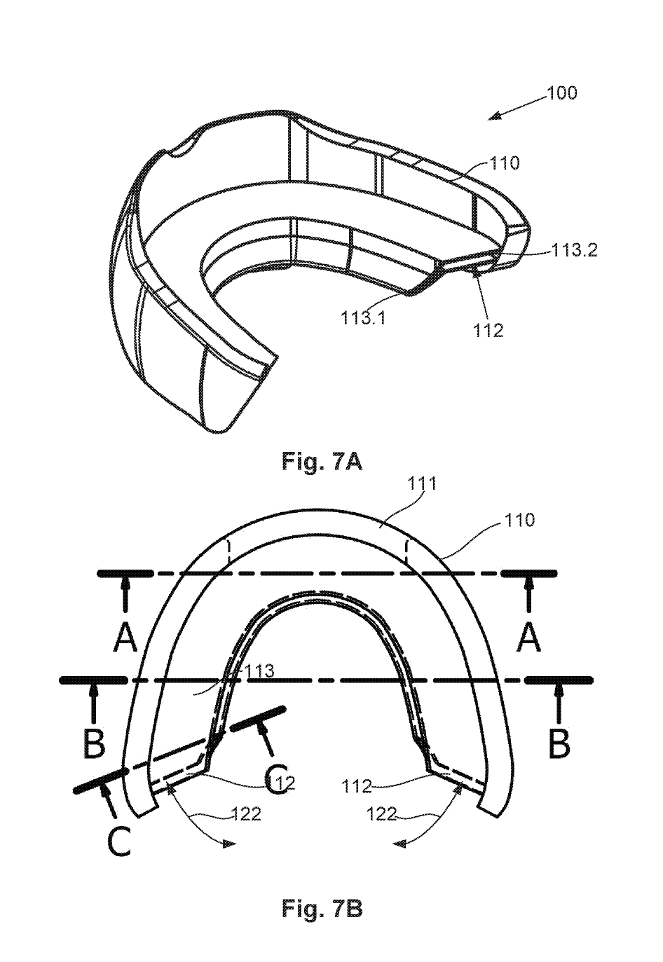

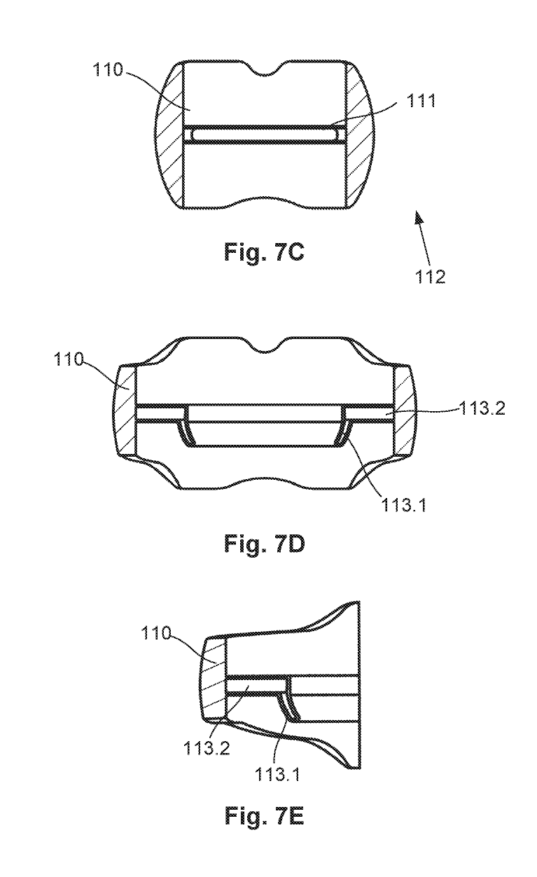

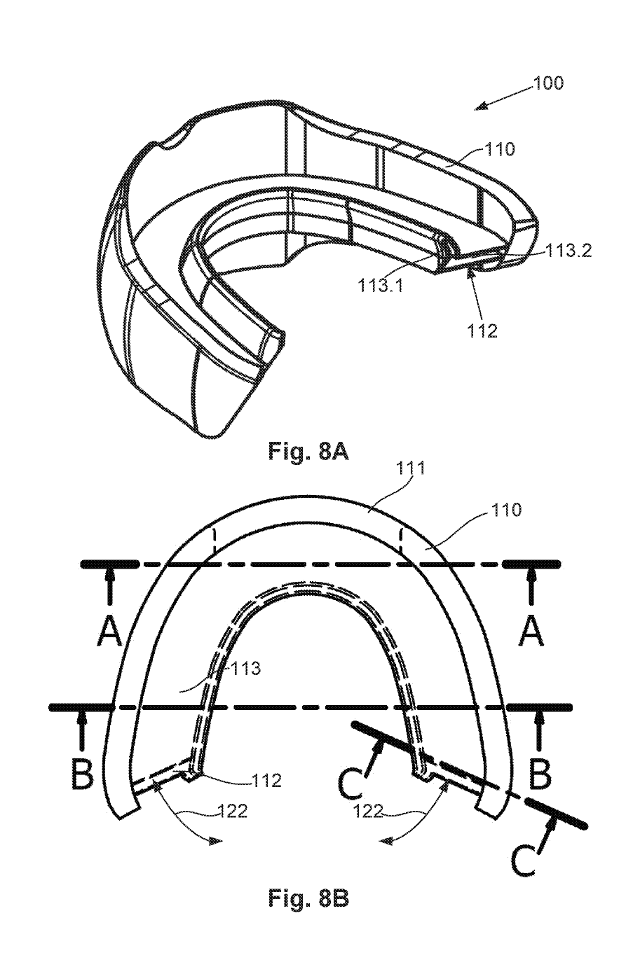

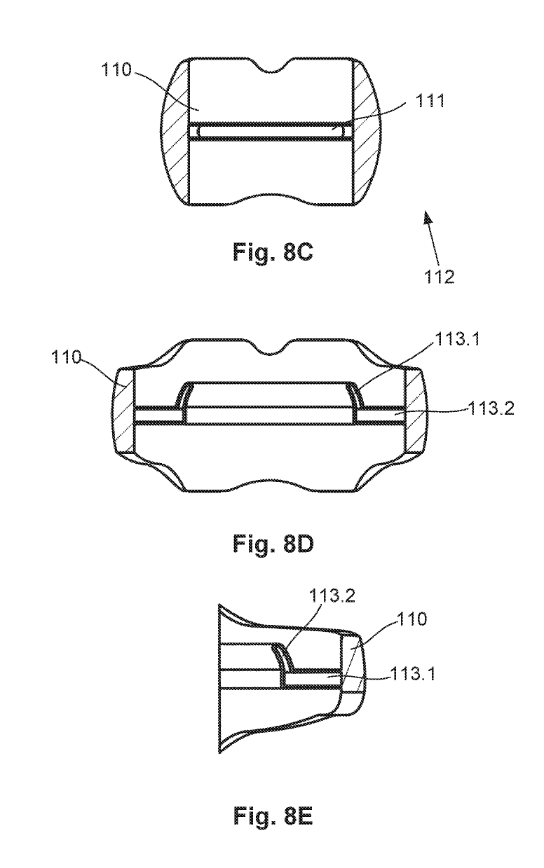

Nonetheless, it will be appreciated that in this configuration, the channel extends between and along an inside of the teeth. In the version of FIGS. 6A to 6G, the first channel portions 113.1 extends both above and below the second channel portion 113.2, whereas in the arrangement of FIGS. 7A to 7E the first channel portion 113.1 extends downwardly only, whilst in the example of FIGS. 8A to 8E the first channel portion 113.1 extends upwardly from the second channel portion 113.2. It will be appreciated that the channel configuration used will vary depending for example on the overall airway cross sectional area required and/or user preference.

It will also be noted that in the examples of in FIGS. 6A to 6G, 7A to 7E and 8A to 8E, the tongue retaining portion is omitted, but this is not essential and the arrangement could be implemented either with or without a tongue retaining portion.

It will be appreciated that such arrangements have not previously been achievable in the art primarily due to the use of molded plastic arrangements which have significant bulk and in particular wall thickness in order to provide sufficient strength to prevent inadvertent crushing of the airway between the user's teeth. However, with the use of titanium printed configurations, this allows a reduced wall thickness to be implemented whilst still providing sufficient strength to prevent crushing. This in turn allows a sufficiently large cross sectional area for an airway that extends substantially between the user's maxillary and mandibular teeth.

Whilst the above described apparatus is particularly useful for controlling sleep apnea and snoring, it is also envisaged that the apparatus may be used for other applications where it is useful to be able to hold and manipulate the tongue. For example, positioning the tongue in a position to maintain an airway may be useful in applications such as anaesthetic recovery and conscious sedation where a user does not necessarily have normal control of their tongue position or airway.

In the above description, the term “additive manufacturing” or “3D printing” is a process of making a three-dimensional solid object of virtually any shape from a digital model. 3D printing is achieved using an additive process, where successive layers of material are laid down in different shapes. 3D printing is also considered distinct from traditional machining techniques, which mostly rely on the removal of material by methods such as cutting or drilling (subtractive processes).

A 3D printer is a limited type of industrial robot that is capable of carrying out an additive process under computer control. To perform a print, the machine reads the design from an STL file and lays down successive layers of liquid, powder, paper or sheet material to build the model from a series of cross sections. These layers, which correspond to the virtual cross sections from the CAD model, are joined or automatically fused to create the final shape. The primary advantage of this technique is its ability to create almost any shape or geometric feature.

Machines, techniques and various parts made from additive manufacturing are now commercially available for various applications in polymers, metals, plaster and ceramics. These include architecture, construction (AEC), industrial design, automotive, aerospace, military, engineering, civil engineering, dental and medical industries, biotech (human tissue replacement), fashion, footwear, jewellery, eyewear, education, geographic information systems, food, and many other fields.

With respect to metals there are a number of variations to the generic term ‘additive manufacturing’ with each machine builder giving their own name to their particular version of the technology. These include wires using Electron Beam Freeform Fabrication (EBF3), granular metals using Direct metal laser sintering (DMLS), Electron-beam melting (EBM), Selective laser melting (SLM) and Selective laser sintering (SLS).

The devices for this invention have been manufactured using Electron Beam Melting using a machine from Arcam AB of Molndal, Sweden. This technique uses powerful electron beams (up to 3500 W) to build up layer-by-layer of metal powders using an ‘Electron Beam Melting’ (EBM) process. The EBM technology is capable of producing complex geometries from defined 3D CAD computer software at speeds up to 80 cm³/hour. Each metal powder layer is melted to the exact geometry defined by the 3D CAD model. The electron beam is managed by electromagnetic coils rather than optics and moving mechanical parts, which is said to allow for very good beam control and extremely fast beam translation. EBM technology also provides a high energy beam which allows for high melting capacity and ultimately high productivity.

Although EBM was used for the device designs proposed herein, other additive manufacturing machines could be used to produce similar designs.

Persons skilled in the art will appreciate that numerous variations and modifications will become apparent. All such variations and modifications which become apparent to persons skilled in the art, should be considered to fall within the spirit and scope that the invention broadly appearing before described.

Thus, for example, it will be appreciated that the airway can also be incorporated into other forms of device, such as existing mandibular advancement devices, including appliances for elastic advancement, advancement with connectors, tongue retaining devices, bimaxillary fixed appliances, bimaxillary occusal appliances, or the like. The above described arrangement is therefore for the purposes of example and is not intended to be limiting.

1.-46. (canceled)

47. Apparatus for providing breathing assistance, the apparatus including:
a. a body for positioning within an oral cavity of a user, the body defining:
i) at least one first opening for allowing airflow between lips of the user;
ii) two second openings provided in the oral cavity to allow air flow into and out of a posterior region of the oral cavity; and,
iii) two channels, each channel connecting a respective second opening to the at least one first opening and each channel passing at least one of at least partially along the buccal cavity and at least partially between the teeth to thereby provide an airway for the user, the airway at least partially bypassing the nasal passage to thereby act to replicate a healthy nasal passage and pharyngeal space; and,
b. a tongue retaining portion including a cavity for receiving a portion of a tongue of the user, in use, wherein the tongue retaining portion is configured to retain the tongue in an extended position to project at least partially between the teeth of the user.

48. Apparatus according to claim 47, wherein retention of the tongue in the cavity is assisted by an at least partial vacuum created within the cavity.

49. Apparatus according to claim 47, wherein at least one of:
a. the tongue retaining portion is integral with the body;
b. the body includes two first openings, each first opening connected to a respective second opening, and wherein the tongue retaining portion is disposed between the two first openings;
c. the tongue retaining portion is secured to the body;
d. at least part of the first opening extends into an anterior portion of the oral cavity and the tongue retaining portion extends at least partially through the first opening;
e. the tongue retaining portion at least partially occludes the first opening so that portions of the first opening disposed about opposing sides of the tongue retaining portion permit airflow into the channels connected to the respective second openings;
f. the tongue retaining portion is sealingly engaged with the body so as to preclude airflow into and out of the anterior portion of the oral cavity; and,
g. the tongue retaining portion is an elliptical bulb structure.

50. Apparatus according to claim 47, wherein each channel at least one of:
a. includes a first channel portion extending through the user's buccal cavity and a second channel portion in fluid communication with the first channel portion and extending between the user's maxillary and mandibular teeth;
b. not contained within the buccal cavity;
c. extends substantially entirely between the user's maxillary and mandibular teeth;
d. includes a first channel portion extending inside of the user's teeth and a second channel portion in fluid communication with the first channel portion and extending between the user's maxillary and mandibular teeth; and,
e. includes a channel portion extending between the user's maxillary and mandibular teeth and no channel portion extending through the user's buccal cavity.

51. Apparatus according to claim 50, wherein a first channel portion extending through the user's buccal cavity has a substantially semi-elliptical cross section and the second channel portion extending between the user's maxillary and mandibular teeth has a substantially rectangular cross section, the second channel portion extending laterally inward from the first channel portion.

52. Apparatus according to claim 50, wherein at least one of the cross sectional shape and cross sectional area of at least one of the first and second channel portions varies at least one of:
a. continuously from the first opening to the second opening; and,
b. in accordance with an anatomy of the oral cavity of the user.

53. Apparatus according to claim 50, wherein the second channel portion has a substantially rectangular cross section extending laterally between the user's maxillary and mandibular teeth in use, and wherein the first channel portion extending inside of the user's teeth has a substantially arcuate shape, extending from an inward end of the second channel portion at least one of:
a. upwardly and inwardly; and,
b. downwardly and inwardly.

54. Apparatus according to claim 47, wherein at least one of:
a. the body is made of at least one of:
i. metal;
ii. titanium alloys;
iii. high strength polymers; and,
iv. cobalt chromium alloys;
b. the body is made using additive manufacturing; and,
c. the body is coated with at least one of:
i. a medical grade polymer;
1. a medical grade elastomer;
2. silicone;
3. polyurethane;
4. epoxy; and,
5. parylene.

55. Apparatus according to claim 47, wherein the apparatus includes at least one insert that is positioned at least partially between the user's teeth and the body in use, and wherein at least one of:
a. the insert is customised for a user's teeth;
b. the insert is at least one of removable and replaceable;
c. the insert is attached to the body using at least one of:
ii. an adhesive; and,
iii. mechanical engagement between the insert and body;
d. the apparatus includes a plurality of inserts for each user, each insert being adapted to provide a different positioning of at least one of the body and the user's teeth.
e. the insert is made of at least one of:
iv. metals;
v. ceramics;
vi. a polymer;
vii. polyether block amide;
viii. polyvinylsiloxane;
ix. polyurethane; and,
x. ethylvinylacetate; and,
f. the insert is made by at least one of:
xi. by additive manufacturing; and,
xii. by laser sintering.

56. Apparatus according to claim 55, wherein the body includes at least one of:
a. at least one side wall extending from a lateral face and a side wall lip projecting inwardly from at least part of the at least one side wall, the at least one insert engaging the lip to thereby couple the insert to the body;
b. a face lip projecting from at least part of a lateral face, the insert engaging the face lip and side wall lip to thereby couple the insert to the body; and,
c. upper and lower faces and corresponding upper and lower side walls, each channel including a first channel portion positioned between an outer side wall and the upper and lower side walls, and a second channel portion extending laterally inwardly from the first channel portion between the upper and lower faces.

57. A method for manufacturing a breathing assistance apparatus for a user, the method including:
a) providing a body for positioning within an oral cavity of the user, the body including:
i. at least one first opening for allowing airflow between lips of the user;
ii. two second openings provided in the oral cavity to allow air flow into and out of a posterior region of the oral cavity; and,
iii. two channels, each channel connecting a respective second opening to a respective first opening and each channel passing at least one of at least partially along the buccal cavity and at least partially between the teeth to thereby provide an airway for the user, the airway at least partially bypassing the nasal passage and acting to replicate a healthy nasal passage and pharyngeal space; and;
b) providing a tongue retaining portion including a cavity for receiving a portion of a tongue of the user, in use, wherein the tongue retaining portion is configured to retain the tongue in an extended position to project at least partially between the teeth of the user, the tongue retaining portion being at least one of part of the body and secured to the body.

58. A method according to claim 57, wherein at least one of:
a) the body is created using additive manufacturing and is made of at least one of:
xiii. metal;
xiv. titanium alloys;
xv. high strength polymers; and,
xvi. cobalt chromium alloys;
b) the method includes applying a coating to the body;
c) the coating is applied to inner surfaces of the body;
d) the method includes applying the coating to the body by at least one of:
xvii. dip coating;
xviii. spray coating; and,
xix. vapour coating;
e) the method includes applying primers to the body prior to coating; and,
f) the method includes polishing at least part of the body using at least one of mechanical and electrochemical polishing.

59. A method according to claim 57, wherein the body is moulded and is made of at least one of:
a) acrylic; and,
b) polymer.

60. A method according to claim 57, wherein the method includes:
a) obtaining shape information indicative of at least one of shape and dimensions of the user's oral cavity using at least one of:
i. an impression;
ii. a series of photos;
iii. a scan;
iv. a CT scan;
v. a 3D scan of the user's teeth;
vi. cone beam imaging; and,
vii. a series of photos of the patient's mouth or impression taken with a smart phone and then loaded into a software program to derive a 3D image including an STL file; and,
b) manufacturing the breathing assist apparatus using the shape information.

61. A method according to claim 60, wherein at least one of:
a) the method includes:
i. selecting one of a number of standard bodies in accordance with the shape information; and,
ii. using the shape information, at least one of:
1. modifying the selected standard body; and,
2. creating at least one insert; and,
b) the method includes:
i. obtaining template data representing a body design;
ii. modifying the body design using the information derived from the scan;
iii. generating modified template data using the modified body design, wherein the modified template data is in the form of a print file for use in an additive manufacturing machine; and,
iv. manufacturing the body using the modified template data.

62. A method according to claim 57, wherein the tongue retaining portion is secured to the body using at least one of an adhesive and mechanical engagement between the insert and body.

63. Apparatus for providing breathing assistance, the apparatus including:
a. a body for positioning within an oral cavity of a user, the body defining:
i. at least one first opening for allowing airflow between lips of the user;
ii. two second openings provided in the oral cavity to allow air flow into and out of a posterior region of the oral cavity; and,
iii. two channels, each channel connecting a respective second opening to a respective first opening and each channel passing at least partially between the teeth and not within the buccal cavity, to thereby provide an airway for the user, the airway at least partially bypassing the nasal passage and acting to replicate a healthy nasal passage and pharyngeal space.

64. Apparatus according to claim 63, wherein each channel includes a first channel portion extending inside of the user's teeth and a second channel portion in fluid communication with the first channel portion and extending between the user's maxillary and mandibular teeth.

65. Apparatus according to claim 64, wherein the second channel portion has a substantially rectangular cross section extending laterally between the user's maxillary and mandibular teeth in use, and wherein the first channel portion extending inside of the user's teeth has a substantially arcuate shape, extending from an inward end of the second channel portion at least one of:
a) upwardly and inwardly; and,
b) downwardly and inwardly.
